(19) United States
(12) Patent Application Publication
(10) Pub. No.: US 2011/0136897 A1
(43) Pub. Date: Jun. 9, 2011

(54) TOLL-LIKE RECEPTOR 9 AGONISTS FOR THE TREATMENT OF ANXIETY-RELATED DISORDERS AND INFLAMMATORY DISORDERS

(75) Inventors: Hermona Soreq, Jerusalem (IL); Gabriel Zimmerman, Jerusalem (IL); Iftach Shaked, Jerusalem (IL)

(73) Assignee: YISSUM RESEARCH DEVELOPMENT COMPANY OF THE HEBREW UNIVERSITY OF JERUSALEM LTD.

(21) Appl. No.: 13/058,876

(22) PCT Filed: Aug. 13, 2009
(86) PCT No.: PCT/IL09/00801

§ 371 Date: Feb. 14, 2011

(30) Foreign Application Priority Data
Aug. 14, 2008 (US) 61088926
Feb. 19, 2009 (US) 61202326

(51) Int. Cl. A61K 31/7084; A61K 31/56; A61K 31/7088

(52) U.S. Cl. 514/44 R; 514/171

(57) ABSTRACT

Uses of TLR-9 agonists are disclosed. The uses include treatment of anxiety-related disorders and inflammatory disorders. For treatment of inflammatory disorders the TLR-9 agonists are administered together with a therapeutically effective amount of a glucocorticoid.

### FIELD AND BACKGROUND OF THE INVENTION

The present invention, in some embodiments thereof, relates to methods of treating anxiety-related disorders and inflammatory disorders by administration of toll-like receptor 9 (TLR-9) agonists.

Psychological stress reactions span cognitive, somatic, emotional and behavioral features. Importantly, major stress regulators modulate immune function. Both post-traumatic stress disorder (PTSD) patients and rodents exposed to acute stressors show elevated levels of pro-inflammatory cytokines (e.g., interleukin (IL)-1β and IL-6) in the serum and brain. Peripheral cytokines can reciprocally access the brain, stimulate their specific receptors at hypothalamic and extra-hypothalamic brain regions and affect neurotransmitter functioning. This spans activation of the hypothalamic-pituitary-adrenal gland (HPA) axis, which induces systemic stress-related symptoms. IL1β is further known to potentiate the consolidation of traumatic memories and the exaggerated anxiety responses to subsequent mild stressors, both characteristic symptoms of PTSD. Thus, pro-inflammatory cytokines serve as body-to-brain messengers accentuating stress reactions.

Peripheral production of pro-inflammatory cytokines is modulated, among other mechanisms, by the family of toll-like receptors (TLRs), pattern recognition receptors of the innate immune system that sense pathogens by recognizing specific viral and bacterial DNA sequence motifs. Yet more specifically, TLR9 is primarily located in the endosomal membrane of dendritic cells (DC), B lymphocytes and monocytes, where it can sense endosomally-internalized oligodeoxynucleotides (ODN) and is activated by some of them. Activated TLR9 can up- or down-regulate cytokines and chemokines production by mechanisms involving the NF-κB and IRF pathways (FIG. 1A). These processes can either accentuate or suppress inflammation. Accordingly, TLR9 modulators have been proposed as therapeutic agents for the treatment of inflammatory disorders including for example asthma, rhinitis, conjunctivitis and inflammatory bowel disease.

Toll-like receptors (TLRs) are type-I transmembrane proteins which are responsible for initiation of innate immune responses in vertebrates. They recognize a variety of pathogen-associated molecular patterns (PAMPS) from bacteria, viruses and fungi and act as a first line of defense against invading pathogens. There are ten human TLRs that elicit overlapping yet distinct biological responses due to differences in cellular expression and signaling pathways they initiate.

Of the ten, TLR3, 7, 8, and 9 are known to localize in endosomes inside the cell and recognize nucleic acids (DNA and RNA) and small molecules such as nucleosides and nucleic acid metabolites. TLR3 and TLR9 are known to recognize nucleic acid such as dsRNA and unmethylated CpG dinucleotide present in viral and bacterial and synthetic DNA, respectively.

U.S. patent application 20060178333 teaches treatment of anxiety e.g., post-traumatic stress disorder (PTSD) with EN101.

U.S. patent application 20060069051 teaches treatment of inflammatory disorders with EN101.

Barrat et al., Immunol Rev. 2008 June; 223:271-83 teaches treatment of autoimmune diseases with TLR9 inhibitors.

### SUMMARY OF THE INVENTION

According to an aspect of some embodiments of the present invention there is provided a method of treating an anxiety-related disorder in a subject in need thereof, the method comprising administering to the subject a therapeutically effective amount of a toll-like receptor 9 (TLR-9) agonist, with the proviso that said TLR-9 agonist is not EN101, thereby treating the anxiety-related disorder.

According to an aspect of some embodiments of the present invention there is provided a method of treating an inflammatory disorder in a subject in need thereof, the method comprising co-administering to the subject a therapeutically effective amount of a glucocorticoid and a TLR-9 agonist, thereby treating the inflammatory disorder.

According to an aspect of some embodiments of the present invention there is provided an article of manufacture comprising a TLR-9 agonist and a glucocorticoid.

According to some embodiments of the invention, the TLR-9 agonist is an oligonucleotide comprising one or more unmethylated CpG dinucleotide (CpG ODNs).

According to some embodiments of the invention, the TLR-9 agonist induces type-1 interferon (IFN1) secretion from dendritic cells.

According to some embodiments of the invention, the TLR-9 agonist upregulates an activity of nuclear factor kappa-light-chain-enhancer of activated B cells (NF-κB).

According to some embodiments of the invention, the CpG ODN is a type-A CpG ODN.

According to some embodiments of the invention, the CpG ODN is a type-B CpG ODN.

According to some embodiments of the invention, the CpG ODN is a type-C CpG ODN.

According to some embodiments of the invention, the administering is effected by systemic administration.

According to some embodiments of the invention, the administering is not effected directly administration to the brain.

According to some embodiments of the invention, the systemic administration is selected from the group consisting of oral administration, intravenous (IV) administration, intrarterial (IA) administration, intramuscular (IM) administration, subcutaneous (SC) administration and intraperitoneal (IP) administration.

According to some embodiments of the invention, the systemic administration is oral administration.

According to some embodiments of the invention, the type-A CpG ODN comprises ODN1585 (SEQ ID NO: 19).

According to some embodiments of the invention, the type-B CpG ODN comprises ODN1826 (SEQ ID NO: 1).

According to some embodiments of the invention, an activity of said TLR-9 agonist is down-regulated by ODN2088 (SEQ ID NO: 2).

According to some embodiments of the invention, the anxiety-related disorder is post traumatic stress syndrome (PTSD).

According to some embodiments of the invention, the TLR-9 agonist comprises ODN1826 (SEQ ID NO: 1).

According to some embodiments of the invention, the therapeutically effective amount of ODN1826 is from about 0.01 μg/kg to 0.09 μg/kg.

According to some embodiments of the invention, the therapeutically effective amount of ODN1826 is about 0.04 μg/kg.

According to some embodiments of the invention, the TLR-9 agonist comprises EN101.

According to some embodiments of the invention, the EN101 comprises hEN101 (SEQ ID NO: 20).

According to some embodiments of the invention, the EN101 is selected from the group consisting of hEN101 (SEQ ID NO: 20), mEN101 (SEQ ID NO: 4) and rEN101 (SEQ ID NO: 21).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-G are schematic diagrams and graphs illustrating that moderate TLR9 activation ameliorates long-term effects of acute stress.

FIG. 1A is scheme showing oligonucleotide internalization to the endosome where it activates toll-like receptor 9 (TLR9). Type-A TLR9 agonists activate interferon regulatory factors (IRFs) inducing type I interferons and activating natural killer cells (NK), macrophages (mφ) and cytotoxic T cells (CTL). Type-B TLR9 agonists activate NF-κB, induce interleukin (IL)-6 and IL-10 secretion and activate B cells. Chloroquine (CQ) inhibits endosomal maturation and the oligonucleotides ODN2088 (SEQ ID NO: 2) and ODN TTAGGG (SEQ ID NO: 3) block TLR9 activation.

FIG. 1B is an overview of the experimental paradigm. Animals that were placed for 10 minutes in a box containing well-soiled cat litter were tested 7 days later in the behavioral elevated plus maze (EPM) and acoustic startle response (ASR) paradigms. Where noted, mice were intraperitoneally injected immediately before the stressor and for a total of 4 consecutive days with the indicated compounds.

FIGS. 1C-F are graphs illustrating the results of the above described experiment. Each circle represents a single mouse. FIG. 1C: Acute TLR9 activation is slightly anxiogenic. On day 7, cat odor exposed saline-injected FVB/N mice (green) showed increased EPM score and acoustic startle response, and decreased habituation to the acoustic pulse, compared with matched controls (orange). Animals treated with 50 μg/kg ODN1826 (SEQ ID NO: 1) (blue) show a slightly anxiogenic behavior compared to saline-treated ones. FIG. 1D: Moderate TLR9 activation is anxiolytic. 0.5 μg/kg ODN1826 (SEQ ID NO: 1) (light blue) treated mice showed lower EPM scores than vehicle-treated animals (green). FIG. 1E: mEN101 (SEQ ID NO: 4) ameliorates stress responses. mEN101 (SEQ ID NO: 4) treatment (50 pg/kg) (yellow) abolished the stress scores of stress-exposed animals in all the measured parameters. FIG. 1F: Antagonizing TLR9 inhibits the TLR9 agonist effect. Co-administration of ODN2088 (SEQ ID NO: 2) with mEN101 (SEQ ID NO: 4) (red) abolished the mEN101-induced amelioration of anxiety-correlated behaviors (yellow).

FIG. 1G is a bar graph illustrating the anxiety Index (the averaged standard scores of EPM index, startle response and startle habituation) of control and experimental groups.

FIGS. 2A-D are graphs illustrating that mEN101 (SEQ ID NO: 4) is a moderate TLR9 activator. A. At 1 μM, mEN101 (SEQ ID NO: 4) activates RAW264.7 cells via TLR9 as revealed by increased nitric-oxide (NO) production. mEN101 (SEQ ID NO: 4) effects are abolished by chloroquine (CQ), as well as by the TLR9 blocking oligonucleotides ODN2088 (SEQ ID NO: 2), and ODN TTAGGG (SEQ ID NO: 3). FIGS. 2B-D: RAW264.7 cells were incubated for 24 hours with the noted TLR9 agonists and inflammatory biomarkers were measured. (FIG. 2B) NO. (FIG. 2C) IL-6. (FIG. 2D) IFN-γ (*: p<0.05).

FIGS. 3A-G are photographs and graphs illustrating that TLR9 activation causes delayed suppression of post-stress c-fos induction. FIG. 3A is an overview of the experimental paradigm. Brain sections were assayed on day 1, 2 hours following acute stress (A) or on day 7, 2 hours following performing in the EPM and ASR paradigms (B). C-fos expression was quantified by counting labeled nuclei. FIGS. 3B-D are photographs illustrating that mEN101 (SEQ ID NO: 4) does not inhibit immediate stress reactions. 2 hours after acute stress exposure, increased c-fos expression was found in the PVN, CA3 and AMG of both untreated (FIG. 3C) and mEN101-treated mice (FIG. 3D) relative to naïve controls (FIG. 3B). Columns: No. of labeled nuclei per section (*: p<0.05, Mann-Whitney U-test). FIGS. 3E-G are photographs illustrating that TLR9 activation by mEN101 (SEQ ID NO: 4) prevents delayed c-fos induction in response to mild stress. On day 7, increased c-fos expression was found in PVN, CA3, AMG of pre-stressed (FIG. 3F) but not stressed and mEN101-treated mice (FIG. 3G) relative to naïve controls (FIG. 3E). (*: p<0.05, Mann-Whitney U-test).

FIGS. 4A-E are graphs and photographs illustrating that TLR9 activation modulates stress-upregulated cytokines in the periphery and stress-related genes and proteins in the brain. FIG. 4A is an overview of the experimental paradigm. On day 4, serum was removed for measuring interleukin (IL)-1β and IL-6 levels (B). On day 7, brain sections were prepared for egr-1 labeling (C-E). FIG. 4B: TLR9 activation by mEN101 (SEQ ID NO: 4) abolishes stress-induced IL1β and IL6 increases (*: p<0.02, Mann-Whitney U-test). FIGS. 4C-E. Increased Egr-1 expression in PVN, CA3 and AMG of stressed mice (FIGS. 4D-E) relative to naïve controls (FIG. 4C). Note that TLR9 activation by mEN101 (SEQ ID NO: 4) (FIG. 4E) abolished such increases in PVN and AMG, but not in CA3. One set of sections out of 3 reproducible ones.

FIGS. 5A-F are graphs illustrating that TLR9 activation modulates anxiety related genes in the brain. Hippocampal RNA was prepared for measuring changes in gene expression on day 4 according to the experimental paradigm described for FIGS. 4A-E. The anxiety-related transcripts GABRA2, CHRNA7, ADCYAP1R1, PKCB, CHRBP and CAMK2A were all down-regulated following stress, up-regulated following mEN101 (SEQ ID NO: 4) injection and reduced to control levels under co-administration of the TLR9 blocker ODN2088 (SEQ ID NO: 2) (*: p<0.05, one-tailed t-test).

FIGS. 6A-F are graphs illustrating that TLR9 −/− mice exhibit accentuated basal anxiety accompanied by subdued stress responses and are refractory to mEN101 (SEQ ID NO: 4). FIG. 6A is an overview of the experimental paradigm. C57/B6 TLR9 −/− mice and strain-matched controls were tested as in FIGS. 1 and 3. FIGS. 6B-D: TLR9 −/− mice show inherited differences in stress responsiveness. Both naïve and stressed TLR9 −/− mice differ from C57/B6 matched controls in their EPM score, startle response and startle habituation. FIG. 6E: Anxiety Index of C57/B6 and TLR9 −/− mice under different experimental conditions. FIGS. 6F-J: TLR9 −/− mice show inherited differences in RT-PCR results for the anxiety-related hippocampal GABRA2, CHRBP, PKCB, CAMK2A and CHRNA7 transcripts, relative to matched controls. (*: p<0.05, one-tailed t-test).

FIGS. 7A-C are graphs illustrating that TLR9 −/− mice are refractory to mEN101. Cat odor exposed and saline-injected TLR9 −/− mice showed increased EPM score and acoustic startle response, and decreased habituation to the acoustic pulse, compared with naïve controls. I.p. injection of 500 μg/kg mEN101 did not affect the reaction of TLR9 −/− mice to the stressor (*: p<0.05, Mann Whitney U test).

FIGS. 8A-N are graphs illustrating that inflammatory markers are correlated with PTSD severity. FIGS. 8A-C: Inflammatory markers are increased in PTSD patients compared to controls matched by age and gender. 8A: IL1β, 8B: IL6. 8C: Inflammatory load (summated IL1β and IL6 levels) in PTSD patients, divided into those with higher and lower loads. FIGS. 8D-I: PTSD behavioral symptoms are increased in patients with high inflammatory load. 8D; Clinician-Administered PTSD Scale (CAPS) re-experience score score. 8E. CAPS arousal score. 8F. Montgomery-Åsberg Depression Rating Scale (MADRS) score. 8G. CAPS. 8H. CAPS avoidance score. 8I. Pittsburgh Sleep Quality Index (PSQI). FIGS. 8J-N: IL1β correlates to PTSD patients hippocampal volume. FIGS. 8J-K: Coronal and saggital X mm image showing the boundaries of the measured structures. FIG. 8L: IL1β is inversely correlated to hippocampal volume of PTSD patients. FIG. 8N: IL1β is not correlated with cerebral cortex volume. (*: p<0.05, one-tailed t-test).

FIG. 9 is a scheme illustrating the proposed mechanism of TLR9 involvement in psychological stress reactins. Psychological stress reactions (orange bent arrow) induce behavioral over-reactions (red bent arrow), adrenal gland hormones secretion and production of the brain signaling IL-1β and IL-6 by peripheral leukocytes, activating receptors located in the hippocampus and amygdale. Consequent activation of the cyngulate cortex and the HPA axis affects the consolidation of fear memories. TLR9 hence modulates psychological stress reactions by monitoring peripherally stress-induced inflammatory responses. Color scale: intensity of response.

FIGS. 10A-E are graphs and images illustrating that TLR9 potentiates gluococorticoid signaling.

FIG. 10A. Scheme: Heat-shock protein (HSP) 90 complexes with glucorticoid receptor (GR) enabling glucocorticoid arrest of inflammation. Upon ligand binding the complex is dissociated and GR binds DNA after homodimerization. FIG. 10B. TLR9 stimulation modulates HSP90 in cell lines and in vivo. RAW264.7 cells were incubated for 24 hours with the indicated oligonucleotides. Mice were exposed to cat odor, intraperitonealy injected with the indicated oligonucleotides for four consecutive days, and sacrificed immediately after the 4^{th} injection. Left: mEN101 up-regulates HSP90β mRNA in RAW264.7 cells. The effect is abolished by the TLR9 antagonist ODN2088 (SEQ ID NO: 2). Right: mEN101 (SEQ ID NO: 4) treatment increases HSP90β transcripts in spleen and frontal lobe. The effect is blocked by co-administration of ODN2088 (SEQ ID NO: 2) (* p<0.05, Mann-Whitney test). FIG. 10C. TLR9 stimulation increases HSP90 and its colocalization with GR in RAW264.7 cells. Cells were incubated for 24 h with 1-10 μM mEN101 (SEQ ID NO: 4). Top: mEN101 (SEQ ID NO: 4) upregulates HSP90, but not GR, in a dose dependent manner. Bottom: HSP90/GR correlation matrices. mEN101 (SEQ ID NO: 4) increases HSP90 and GR signal colocalization in a dose-dependent manner. Quantification: HSP90 and GR signals correlation, HSP90 instances given GR, and GR instances given HSP90. FIG. 10D. TLR9 activation increases dexamethasone binding. A whole cell binding assay was used to determine GR number and affinity. RAW264.7 and C2C12 cells were grown in 24-wells. Treated cells were incubated for 24 h with μM mEN101 (SEQ ID NO: 4). The medium was replaced with 0.5-40 nM [³H]dexamethasone (85.0 Ci/mmol) for scatchard analysis or with 40 nM for whole cell binding assay. After 2 hours incubation cells were washed 6 times with cold PBS and lysed in 1M NaOH. Lysates were harvested and counted in a beta-spectrometer. Left: In TLR9-expressing RAW264.7 cells mEN101 increases whole cell [³H]dexamethasone binding in a dose dependent manner (* p<0.05, Mann-Whitney test). Center: In RAW264.7 cells, TLR9 activation by 10 μM mEN101 (SEQ ID NO: 4) increases GR Bmax, without altering its Kd. Right: the same treatment yields no effect in C2C12 cells, which do not express TLR9. FIG. 10E. TLR9 activation increases corticosterone anti-inflammatory effect. Starved RAW264.7 cells were incubated with different concentrations of ODN1826 (SEQ ID NO: 1). After 24 hours 1 μg/ml LPS with or without 10 (left) or 50 μM (right) corticosterone were added to the medium. Nitric oxide (NO) was measured after 24 hours. At low doses (0.01-1 nM), ODN1826 (SEQ ID NO: 1) potentiated CORT anti-inflammatory effect. However, at higher doses (10-100 nM) ODN1826 (SEQ ID NO: 1) increased cell activation, probably due to its inflammatory effect.

### DESCRIPTION OF SPECIFIC EMBODIMENTS OF THE INVENTION

The present invention, in some embodiments thereof, relates to methods of treating anxiety-related disorders and inflammatory disorders by administration of toll-like receptor 9 (TLR-9) agonists.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Peripheral production of pro-inflammatory cytokines is modulated, among other mechanisms, by the family of toll-like receptors (TLRs), pattern recognition receptors of the innate immune system that sense pathogens by recognizing specific viral and bacterial DNA sequence motifs.

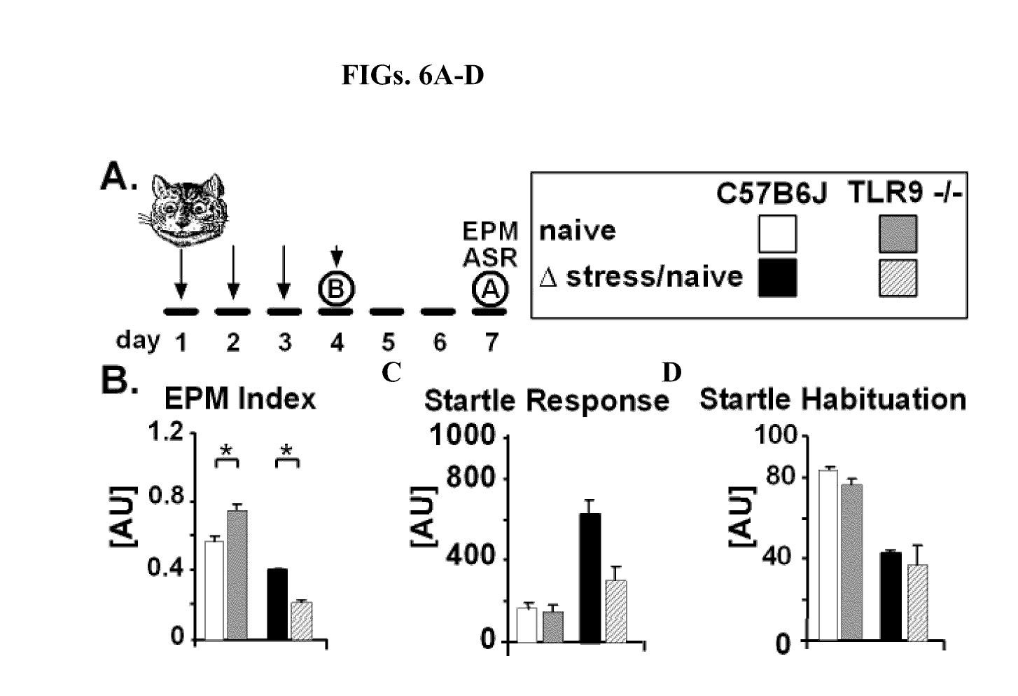

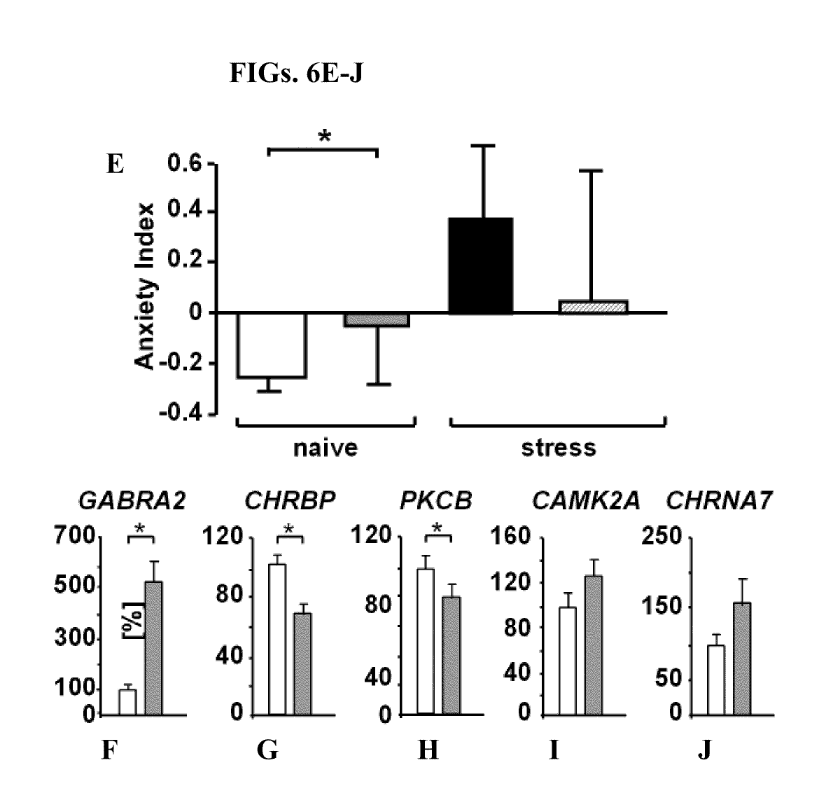

The present inventors have now uncovered that TLRs (and more specifically TLR-9) are also involved in stress-induced immune responses. Specifically, the present inventors showed that genomic ablation of TLR9 increased basal anxiety while reducing reactiveness to acute stressors and inducing stress-associated changes in hippocampal gene expression (FIGS. 6A-J).

The present inventors thus propose the use of TLR-9 agonists for the treatment of anxiety-related disorders such as PTSD.

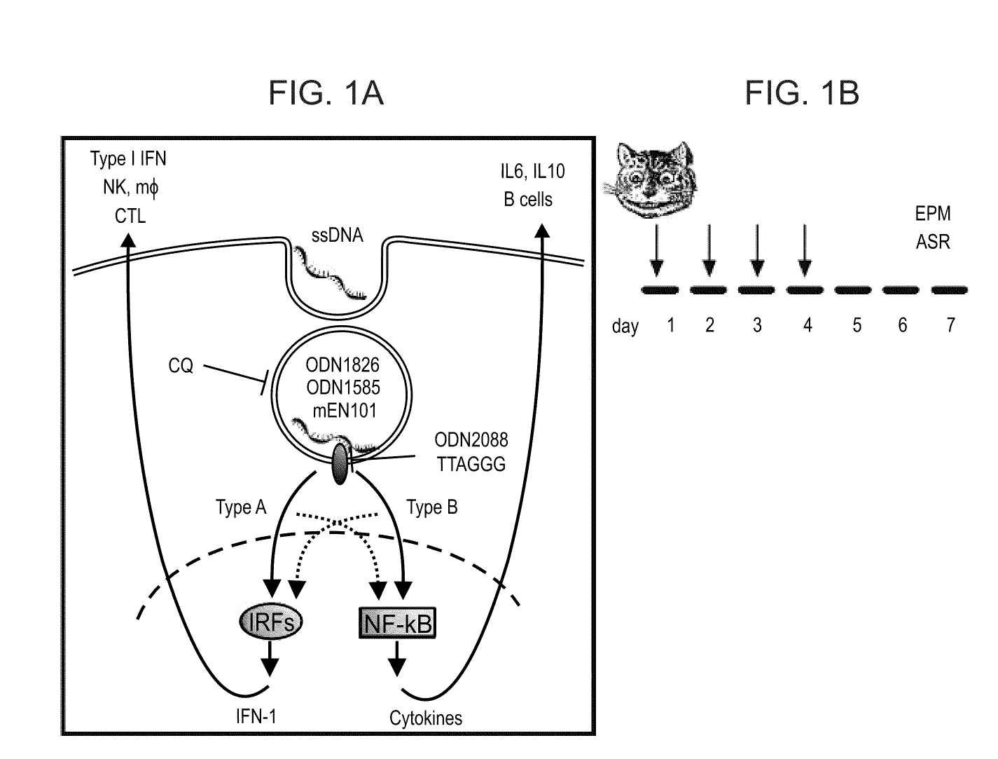

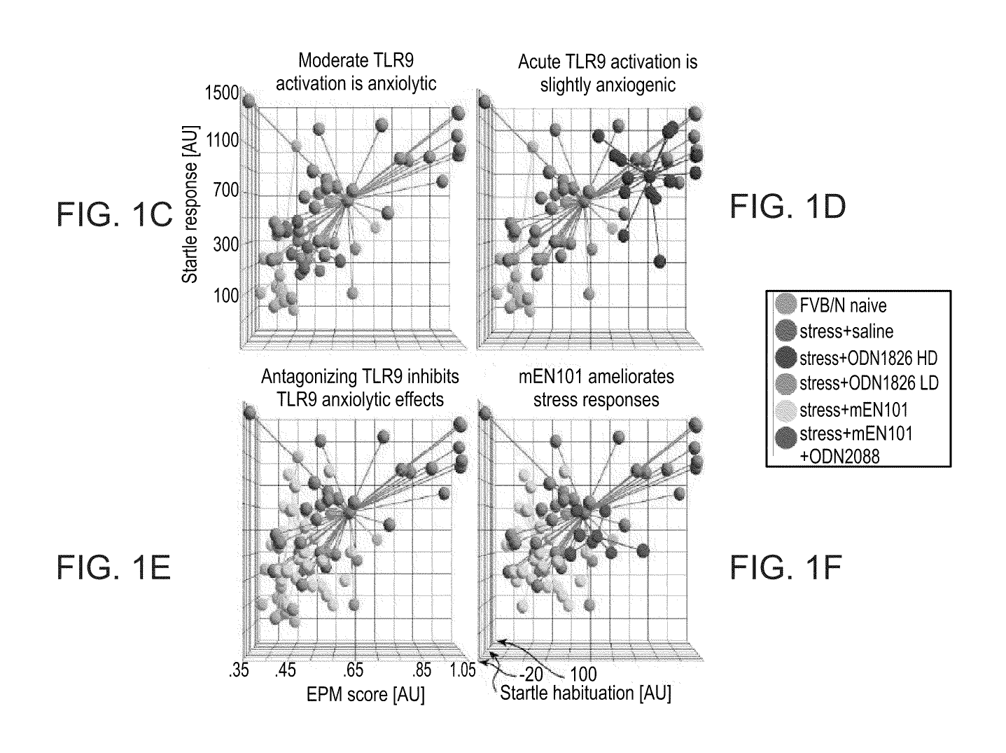

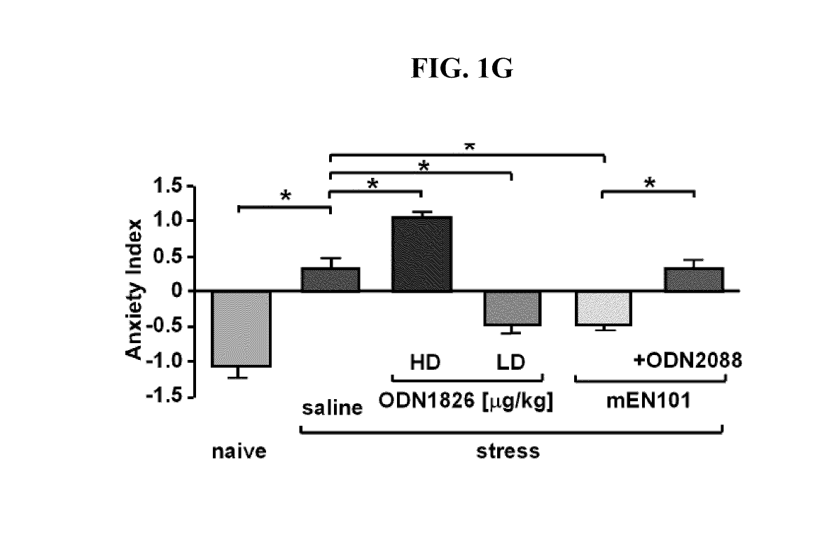

Whilst reducing the present invention to practice, the present inventors showed that peripheral administration of ODN1826, an established TLR9 activator could, at low levels, down-regulate the stress levels of mice subjected to predator odor (FIGS. 1C-D and FIG. 1G). In addition, the present inventors showed that the effect of peripheral administration of mEN101 (a known stress-suppressing oligonucleotide) on the stress levels of mice subjected to predator odor, could be blocked by TLR-9 antagonists (FIGS. 1E-G).

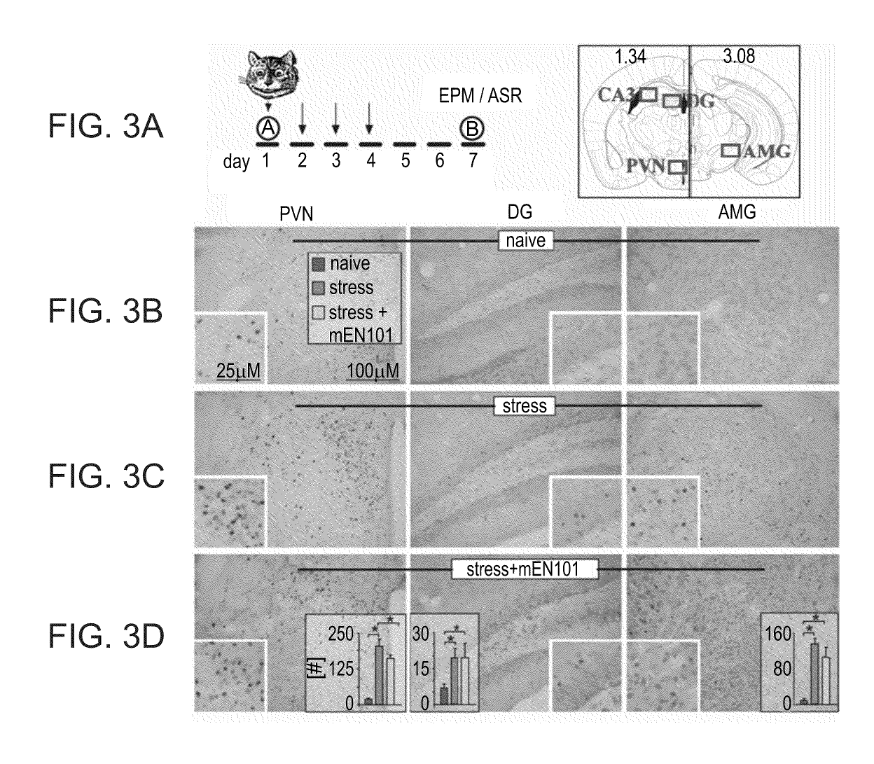

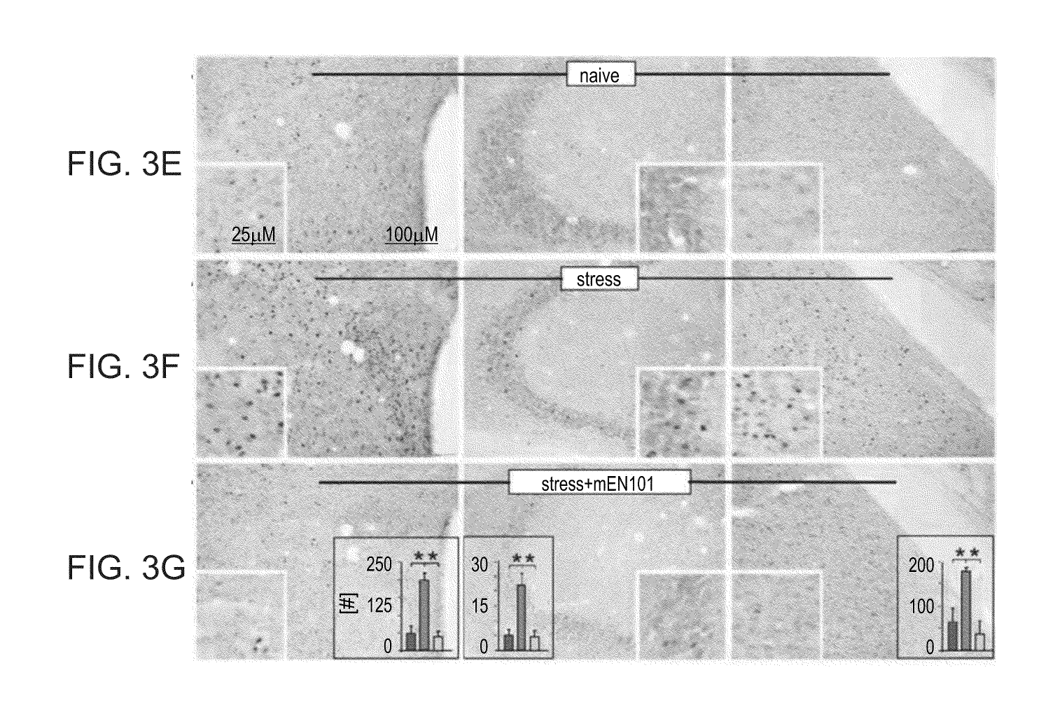

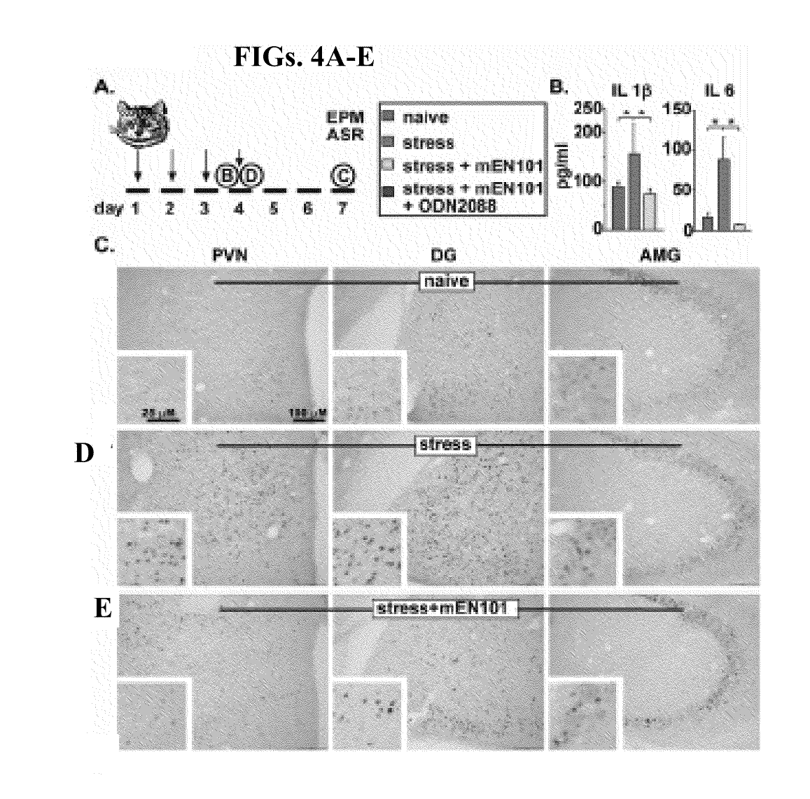

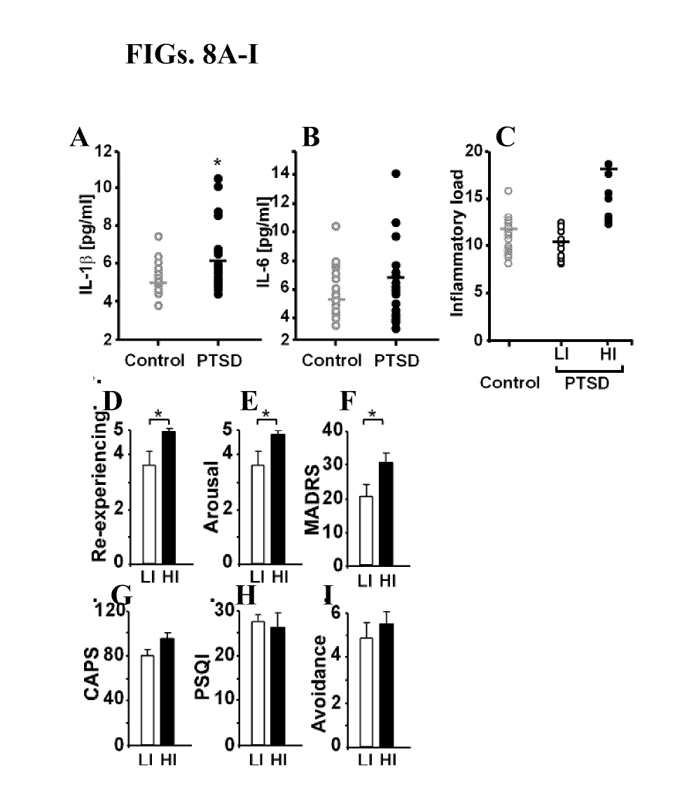

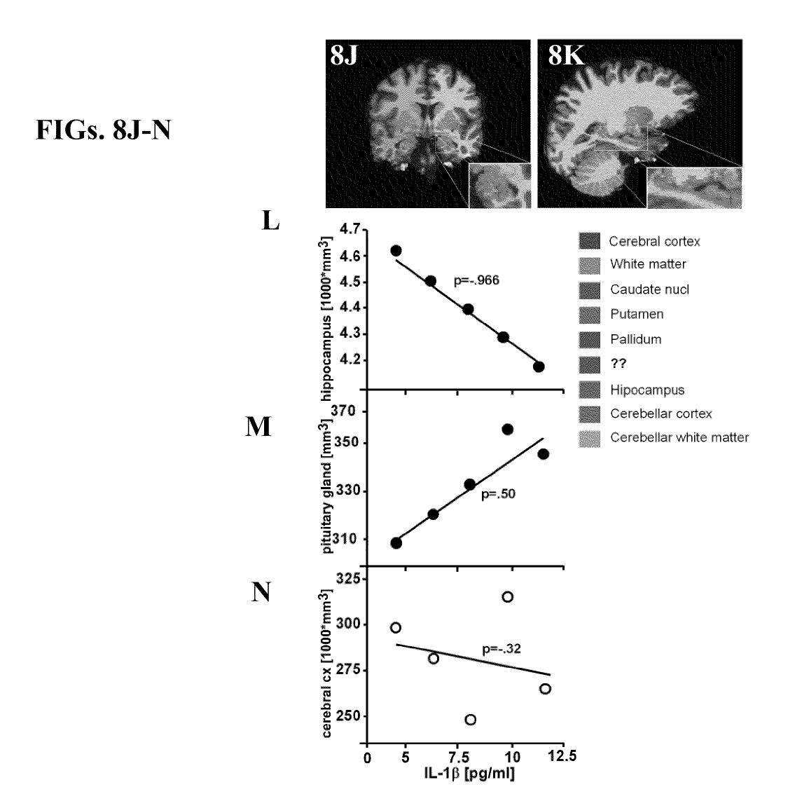

The present inventors further showed that transient and moderate TLR9 activation exerts long-lasting suppression of two pro-inflammatory cytokines shown to be up-regulated in the serum of post-traumatic syndrome (PTSD) patients—IL-1 and IL-6 (FIGS. 4B and 8A-B). The suppression of pro-inflammatory cytokines was shown to be accompanied by blockade of stress-inducible activities in responding brain nuclei (FIGS. 3A-D and 4A-D).

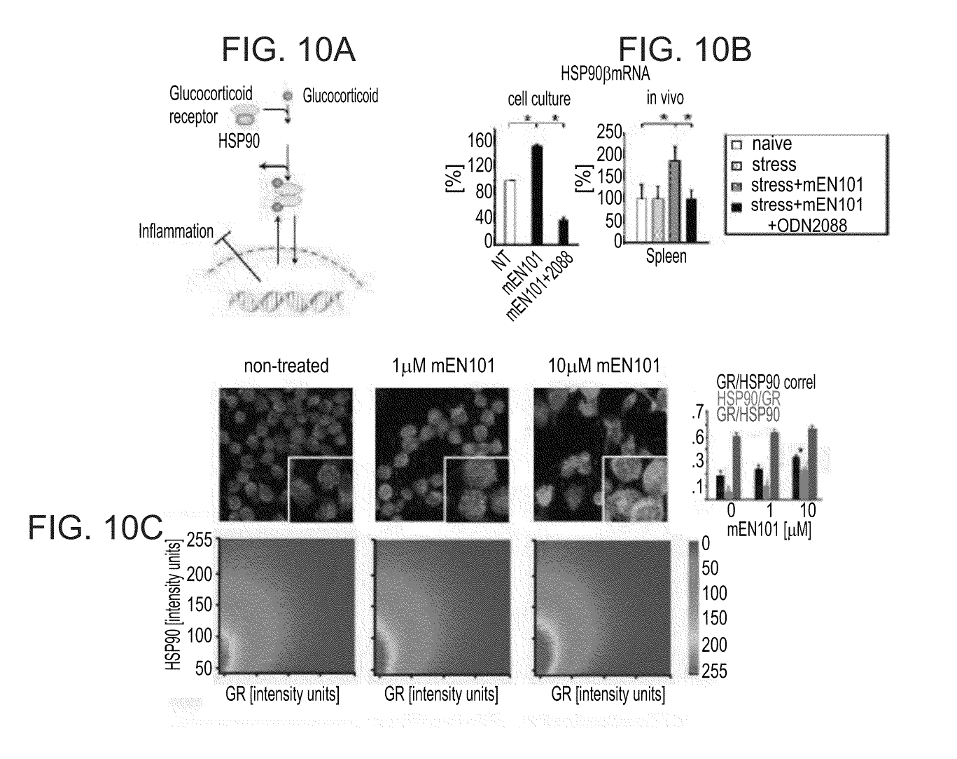

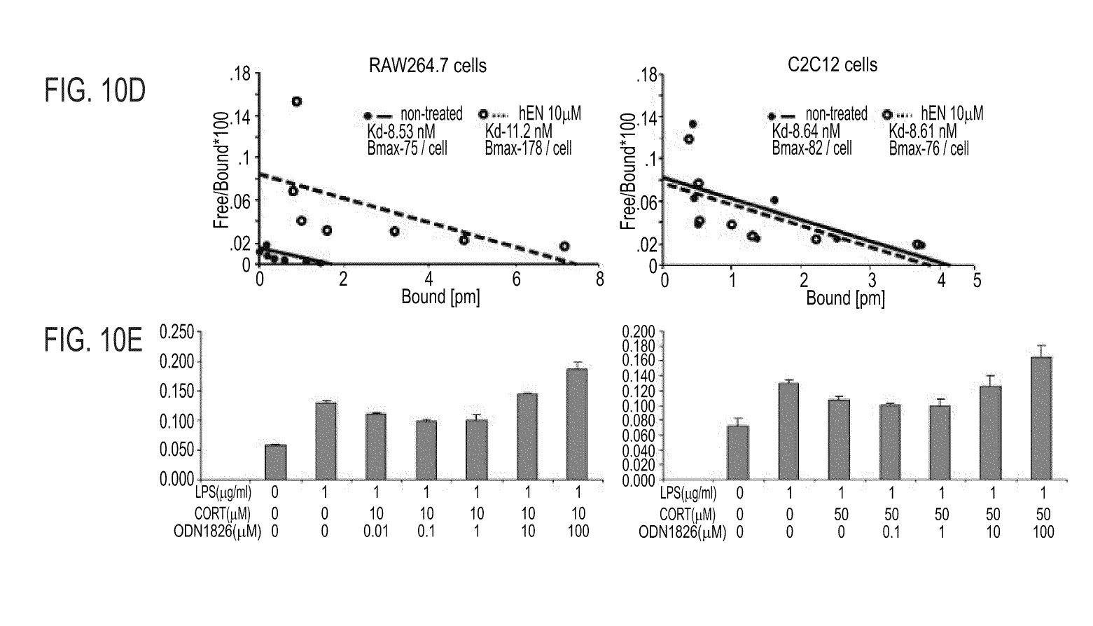

In addition, the present inventors showed that pre-treating TLR-9-expressing cells with TLR-9 agonists increase glucocorticoid (GC) binding to glucocorticoid receptor (GR) thereby potentiating GC anti-inflammatory effects. Accordingly, the present inventors propose that TLR9 agonists can be used to potentiate GC anti-inflammatory effect in vivo (FIGS. 10B-E).

Thus according to one aspect of the present invention there is provided a method of treating an anxiety-related disorder in a subject in need thereof, the method comprising administering to the subject a therapeutically effective amount of a toll-like receptor 9 (TLR-9) agonist, with the proviso that said TLR-9 agonist is not EN101, thereby treating the anxiety-related disorder.

As used herein the phrase “anxiety-related disorder” relates to a psychological and physiological state characterized by cognitive, somatic, emotional, and behavioral components. These components combine to create the painful feelings typically recognized as anger, fear, apprehension, or worry. Anxiety is often accompanied by physical sensations such as heart palpitations, nausea, chest pain, shortness of breath, stomach aches, or headache. Anxiety disorders may be the result of amounts of stress that stretches or exceeds the subject's adaptive capacity.

Examples of anxiety-related disorders include, but are not limited to panic disorders, phobias, obsessive-compulsive disorder, post-traumatic stress disorder (PTSD) and separation anxiety.

According to one embodiment, the anxiety-related disorder is PTSD.

PTSD is a disorder which results from a traumatic experience. Post-traumatic stress can result from an extreme situation, such as combat, rape, hostage situations, or even serious accident. It can also result from long term (chronic) exposure to a severe stressor, for example soldiers who endure individual battles but cannot cope with continuous combat. Common symptoms include flashbacks, avoidant behaviors, and depression.

Typically, the subject of this aspect of the present invention is a mammalian subject, for example human.

The phrase “toll-like receptor 9 agonist” or “TLR-9 agonist” as used herein refers to a compound or substance that binds to a TLR9 receptor and induces a signaling event mediated by a TLR9 signal transduction pathway.

Thus for example the TLR-9 agonist may be one that binds to the TLR-9 receptor and induces type-1 interferon (IFN1) secretion from dendritic cells via interferon regulatory factor (IRF) 7-mediated pathways, activating natural killer cells, macrophages and cytotoxic T cells. Alternatively, or additionally, the TLR-9 agonist may be one that binds to the TLR-9 receptor and activate NF-κB transcription pathways, leading to the production of cytokines such as IL4, -6, -10 and TNF-α and inducing B cell maturation and antibody secretion.

It will be appreciated that an activity of a TLR-9 agonist is down-regulated by a TLR-9 antagonist. Thus, for example the TLR-9 agonists of this aspect of the present invention are typically down-regulated by ODN2088 (SEQ ID NO: 2), a known TLR-9 antagonist.

According to one embodiment, the TLR-9 agonist is an oligonucleotide-based compound. The oligonucleotide may be from a natural source ((bacteria, virus) or synthetic (immune modulatory oligonucleotides (IMOs)).

Exemplary oligonucleotide-based TLR-9 agonists are those that comprise one or more unmethylated CpG dinucleotide (“CpG ODNs”). In non-limiting embodiments of the invention, such oligonucleotides may contain phosphorothioate linkages (at some or all bonds) and/or 2-o-methyl 3′ terminal blockade or other modifications which improve stability, uptake, etc.

A number of CpG ODNs that activate TLR9 are known in the art. Some are species specific.

Human CpG ODNs have been divided into three types, as follows: Type A (D) CpG ODNs, which have polyG motifs with phosphohorothioate linkages at the 5′ and 3′ ends and a PO-containing palindrome CpG-containing motif at its center--these are strong inducers of IFN-alpha production by plasmacytoid dendritic cells and are potent NK cell activators; Type B (K) CpG ODNs, which have a full phosphorothioate backbone with one or more CpG motifs without polyG; they are potent activators of B cells but weaker inducers of IFN-alpha production; and Type C CpG ODNs, which have a complete phosphorothioate backbone without polyG, but have CpG motifs and palindromes; they produce A and B-like effects (stimulate IFN-alpha and B cells).

Either type A, type B or type C CpG ODNs may be used according to this aspect of the present invention. Non-limiting example of CpG ODNs which may be used according to the invention include, but are not limited to, 5′-TCCATGACGTTCCTGACGTT (SEQ ID NO:1; ODN 1826, Invivogen, San Diego, Calif.), 5′-ggGGTCAACGTTGAgggggg (SEQ ID NO: 19; ODN 1585, Invivogen, San Diego, Calif.), 5′-TCG TCG TTT TGT CGT TTT GTC GTT-3′ (SEQ ID NO:22; CpG ODN 2006, InvivoGen, San Diego, Calif.), CpG ODN 2006-G5 (InvivoGen, San Diego, Calif.), 5′-GGG GGA CGA TCG TCG GGG GG-3′ (SEQ ID NO:23; CpG ODN 2216, InvivoGen, San Diego, Calif.), 5′-TCG TCG TCG TTC GAA CGA CGT TGA T (SEQ ID NO:24; CpG ODN M362, InvivoGen, San Diego, Calif.), 5′-TCG TCG TTT TGT CGT TTT GTC GTT-3′ (SEQ ID NO:25; CpG ODN 7909, Coley Pharmaceutical Group, Ottawa, Ontario, Canada), D(5′-TCTGTCGTTCT-X-TCTTGCTGTCT-5) (SEQ ID NO:26) where X is a glycerol linker (Idera Pharmaceuticals, Cambridge, Mass.; see Putta et al., Nucl. Acids Res. 34(11):3231-3238), d(5′-TCTGTC*GTTCT-X-TCTTGC*TGTCT-5′) (SEQ ID NO:27) where C*=N³-Me-dC and X is a glycerol linker (Idera Pharmaceuticals, Cambridge, Mass.; see Putta et al., Nucl. Acids Res. 34(11):3231-3238), and d(5′-TCTGTCG*TTCT-X-TCTTG*CTGTCT-′) (SEQ ID NO:28) where G*=N′-Me-dG and X is a glycerol linker (Idera Pharmaceuticals, Cambridge, Mass.; see Putta et al., Nucl. Acids Res. 34(11):3231-3238). An exemplary type C CpG ODN is SD101 (Dynavax Technologies Corporation).

For additional TLR9 agonists, see Daubenberger, 2007, Curr. Opin. Molec. Ther. 9:45-52 and Krieg, 2006, Nat. Rev. Drug Disc. 5:471-484 and U.S. Patent Application No. 20090053206, incorporated herein by reference.

In further embodiments, the TLR-9 agonists used to treat anxiety-related disorders are CpG ODNs which are at least 90 percent and preferably at least 95 percent homologous (e.g. identical) to any of the CpG ODNs referred to herein (where homology may be determined by standard software such as BLAST or FASTA).

In non-limiting embodiments of the invention, a mixture of two or more TLR-9 agonists may be used for the treatment of anxiety-related disorders.

TLR-9 oligonucleotide agonists designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

Methods of screening for TLR-9 agonists are known in the art—see for example U.S. Pat. Application No. 20060269936 and U.S. Pat. Application No. 20030104523, both of which are incorporated herein by reference.

As mentioned, in order to treat an anxiety-related disorder, the TLR-9 agonists of this aspect of the present invention are provided in a therapeutically effective amount—i.e. in an amount effective to prevent, alleviate or ameliorate symptoms of the anxiety-related disorder (e.g., PTSD).

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually such that blood levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

For each TLR-9 agonist a dose is typically selected that does not induce an immunogenic response—e.g. elevation of IL-6 and/or IL-1 above basal levels. Thus, for example a therapeutically effective amount of a TLR-9 agonist is an amount which is about half an amount, one tenth of an amount or one hundredth of an amount that will induce a TLR-9-dependent elevation in serum levels of IL-6 and/or IL-1 in that subject. It will be appreciated that for each TLR-9 agonist, the amount that induces an immune response will be different. Accordingly, the therapeutically effective amount of each agonist should be determined on an individual basis.

The dose can be formulated in animal models to achieve a desired concentration or titer or a desired effect. Thus, for the treatment of anxiety related disorders, a dose can be selected that shows an anxiolytic effect in mouse models of anxiety. These models include exposing mice to soiled cat litter and testing them several days later on an elevated plus maze (EPM) or using the acoustic startle response (ASR) paradigm. Such information can be used to more accurately determine useful doses in humans.

For ODN1826 the amount in humans should be the human equivalent of the therapeutically effective amount in mice—namely about 0.5 μg/kg. The human equivalent may calculated based on a number of conversion criteria as explained below; or may be a dose such that either the plasma level will be similar to that in the murine following administration at a dose as specified above; or a dose that yields a total exposure (namely area under the curve—AUC—of the plasma level of said agent as a function of time) that is similar to that in murine at the specified dose range.

It is well known that an amount of X mg/Kg administered to mice can be converted to an equivalent amount in another species (notably humans) by the use of one of possible conversions equations well known in the art. According to one conversion, 0.5 μg/Kg in mice is equivalent to about 0.04/Kg in humans; assuming an average weight of 70 Kg, this would translate into an absolute dosage of about 2 μg of ODN1826.

Thus, the present inventors contemplate providing ODN1826 at a range from about 0.001 μg/kg to 0.09 μg/kg, more preferably from 0.01 μg/kg to 0.09 μg/kg and more preferably from 0.01 μg/kg to 0.06 μg/kg. Depending on the severity and responsiveness of the condition to be treated, dosing can be of a single or a plurality of administrations, with course of treatment lasting from several days to several weeks or until cure is effected or diminution of the disease state is achieved.

Since the present inventors showed that the anxiolytic effect of the TLR-9 agonists of the present invention took place peripherally and not centrally, contemplated modes of administration of the TLR-9 agonists of the present invention include any mode of systemic administration that does not involve direct administration to the central nervous system (CNS). These include for example oral administration, intravenous (IV) administration, intrarterial (IA) administration, intramuscular (IM) administration, subcutaneous (SC) administration and intraperitoneal (IP) administration.

The TLR-9 agonists of this aspect of the present invention may be provided per se, or may be or in a pharmaceutical composition where they are mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the TLR-9 agonist accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

According to this aspect of the present invention, the TLR-9 agonist is not formulated for crossing the blood brain barrier.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

In accordance with a preferred mode of the invention the treatment is either preventive—to be given before a possible traumatic state is expected to occur (such as for example before an operation, before going to battle).

In accordance with another preferred option the treatment is given as an early intervention after a traumatic incident, known as Critical Incident Stress Management (CISM) and is used to attempt to reduce traumatic effects of an incident, and potentially prevent a full-blown occurrence of PTSD. Such treatment should be given shortly, preferably in the first few days, following the traumatic event (terrorist attack, earthquake, rape etc.) in order to prevent/lessen the consolidation of memories of the traumatic event that can later develop to a full PTSD.

As mentioned, the present inventors found that TLR-9 agonists increase glucocorticoid (GC) binding to glucocorticoid receptor (GR) and potentiate GC anti-inflammatory effect in leukocytes. Excessive glucocorticoid levels, resulting from administration as a drug, have side-effects on many systems, some examples including inhibition of bone formation, suppression of calcium absorption and delayed wound healing. Accordingly, the present inventors propose lowering the effective clinical dose of the glucorticoid by co-administration with a TLR-9 agonist.

Thus, according to another aspect of the present invention there is provided a method of treating an inflammatory disorder in a subject in need thereof, the method comprising co-administering to the subject a therapeutically effective amount of a glucocorticoid and a TLR-9 agonist, thereby treating the inflammatory disorder.

The term “glucocorticoid” refers to a member of the family of hormones that predominantly affects the metabolism of carbohydrates and, to a lesser extent, fats and proteins (and have other effects). Glucocorticoids are made in the peripheral part (the cortex) of the adrenal gland and are chemically classed as steroids. Cortisol is the major natural glucocorticoid. Nonetheless, the term glucocorticoid also applies to equivalent hormones synthesized in the laboratory.

A non-limiting list of glucocorticoids may be found at the internet site wwwdotsteraloidsdotcom/, incorporated herein in its entirety by reference. Examples include prednisolone hemisuccinate, methylprednisolone heeimisuccinate, dexamethasone hemisuccinate, allopregnanolone hemisuccinate; beclomethasone 21-hemisuccinate; betamethasone 21-hemisuccinate; boldenone hemisuccinate; prednisolone hemisuccinate, sodium salt; prednisolone 21-hemisuccinate; nandrolone hemisuccinate; 19-nortestosterone hemisuccinate; deoxycorticosterone 21-hemisuccinate; dexamethasone hemisuccinate; dexamethasone hemisuccinate spermine; corticosterone hemisuccinate; cortexolone hemisuccinate.

The TLR-9 agonists according to this aspect of the present invention include all the TLR-9 agonists described herein above and further EN101 oligonucleotides. EN101 oligonucleotides are antisense oligonucleotide targeted against human, rat or mouse (hEN101, rEN101 or mEN101, respectively) AChE mRNA—see U.S. Patent Application 20060178333, incorporated herein by reference. Exemplary sequences of EN101 include the human EN101 (hEN101; SEQ ID NO: 20), mouse EN101 (mEN101; SEQ ID NO: 4) or rat EN101 (rEN101; SEQ ID NO: 21).

The combination of TLR-9 agonist and glucocorticoid is preferably utilized for the treatment or prevention of any disease whose acceptable form of treatment includes administration of glucocorticoids. These include for the treatment or prevention of inflammatory diseases, neurodegenerative diseases and the treatment or prevention of cancer which are known to be sensitive to steroids, such as cancers of haematopoeitic origin including lymphoma, leukemia, myeloma, breast cancer and prostate cancer.

Inflammatory diseases—Include, but are not limited to, chronic inflammatory diseases and acute inflammatory diseases.

Inflammatory Diseases Associated with Hypersensitivity

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza KM. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann NY Acad Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arthritis, Takayasu's arthritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like beta-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann NY Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

Autoimmune Diseases

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S 107-9), Wegener's granulomatosis, Takayasu's arthritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann NY Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann NY Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

As used herein, the term “co-administer” refers to administering more than one pharmaceutical agent to a patient. In certain embodiments, co-administered pharmaceutical agents are administered together in a single dosage unit (i.e. in a single formulation). In certain embodiments, co-administered pharmaceutical agents are administered separately. In certain embodiments, co-administered pharmaceutical agents are administered at the same time. In certain embodiments, co-administered pharmaceutical agents are administered at different times.

Thus, the present invention contemplates pharmaceutical compositions, such as those described herein above, comprising both the TLR-9 agonist and the gluocorticoid. In addition, the present invention contemplates articles of manufacture comprising both the TLR-9 agonist and the gluocorticoid. The TLR-9 agonist and the gluocorticoid in the article of manufacture may be packaged in individual wrappers or together in the same wrapper.

The amount of TLR-9 agonist that is capable of enhancing the activity of a glucocorticoid can be determined from in-vitro assays and animal models. According to one embodiment the TLR-9 agonist is administered to a subject at an amount which does not cause an inflammatory effect, such as the up-regulation of serum levels of interleukin-6 (IL-6) or interleukin-1 (IL-1).

Since the TLR-9 agonists of the present invention potentiate the effects of the glucocorticoids, it will be appreciated that the glucocorticoids are administered at a dose which is typically less than that than if the TLR-9 agonists were not present.

Contemplated modes of administration of the TLR-9 agonists for potentiating the effect of a glucocorticoid are the same as those described herein above, except that in this case CNS delivery of the TLR-9 agonists are also contemplated.

Efficacy and safety studies of systemic administration of glucocorticoids, revealed that in addition to the profound activity of the drug in many different tissues, these drugs have rapid clearance from plasma thereby requiring high and frequent dosing to obtain effective amounts at the target site. Thus, typically glucocorticoids are administered locally, (e.g. by the use of inhalers in asthma and in intraarticular injection in arthritis), although all other modes of administration, such as those described herein above are contemplated.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Methods for Examples 1-7

Cell cultures: Spleens were removed from 5-6 week old FVB/N mice (Harlan Laboratories, Israel) and mashed until a homogeneous cell suspension was obtained. The suspension was centrifuged in a LSM Lymphocyte Separation Medium (MP Biomedicals, Solon, Ohio) and the mononuclear cell layer was separated. Cells were adjusted to a 5×10⁶ cells/ml and cultured in RPMI 1640 (Biological Industries, Israel) supplemented with 5% FCS, 2 nM L-glut, 100 μg/ml streptomycin, 100 U/ml penicillin and 5×10⁻⁵ 2-mercaptoethanol (Sigma, Israel).

The mouse macrophage cell line RAW 264.7 (ATCC, Bethesda, Md.) was cultured in Dulbecco's modified Eagle's medium (DMEM) (Biological Industries) supplemented with 10% heat-inactivated fetal bovine serum (Biological Industries, Israel), 100 μg/ml streptomycin, 100 U/ml penicillin (Sigma) and 2 mM 1-glutamine (Biological Industries) at 37° C. in a humidified atmosphere with 5% CO₂.

Determination of nitric oxide (NO) production: Nitrite concentration in the medium of cultured cells was measured as an indicator of NO production according to the Griess reaction. 1×10⁵ mouse RAW264.7 cells were grown in 0.5 ml medium in 48-wells plate until 80% confluence was obtained. 100 ml supernatant was incubated with 100 ml Griess reagent (1% sulfanilamide, 0.1% naphthylethylenediamine dihydrochloride, 5% H₃PO₄). Absorbance was read at 540 nm after 10 min using SPECTRAFluor Plus spectrophotometer (TECAN, UK).

ELISA: Serum was extracted by centrifuging blood in heparin-containing Microtainer LH vials (BD, Franklin Lakes, N.J.) for 10 minutes at 3000 g. Serum and cell-culture supernatants were analyzed for IL-1β, IL-6, TNF-α and IFN-γ using respective ELISA Ready-SET-Go (eBioscience, San Diego, Calif.) kits as instructed. Intensities were read using a Tecan SPECTRAFluor Plus spectrophotometer (Mannedorf, Switzerland).

Animals: FVB/N or adult TLR9 −/− mice and strain-matched wild type C57/B6J mice were housed four per cage, with constant temperature (21±1° C.), a 12 hour light/dark cycle, free access to food and water and habituation to the housing conditions by daily handling. Experiments were performed 1 week after arrival in the animal facility.

Behavioral paradigms: Mice were placed for 10 minutes on well-soiled cat litter (in use by the cat for 2 days, sifted for stools). One week after exposure, animals were tested according to Cohen et al. (2005) Biol Psychiatry 58, 640 in the elevated plus maze (EPM) and the acoustic startle response (ASR) paradigms. The EPM is a plus-shaped platform with two opposing open arms and two opposing closed arms (surrounded by 14-cm high opaque walls on three sides). Mice were placed on the central platform and were allowed to explore the maze for 5 min. An E, was calculated according to

$$E P M Anxiety Index = 1 - [ ( \frac{time spent in open arms}{total time on maze} ) + ( \frac{no . of entries to open arms}{total no . of entries} ) ] / 2$$

Startle response was measured using two ventilated startle chambers (SR-LAB System, San Diego Instruments, San Diego, Calif.). Each Plexiglas cylinder rests on a platform inside a sound-attenuated, ventilated chamber. Startle sessions started with a 5-minute acclimatization period to a background of 68 dB white noise, followed by 30 acoustic startle stimuli (110 dB white noise of 40 ms duration with 30 s or 45 s inter-trial interval). Startle amplitude was averaged over all 30 trials, and percent habituation was calculated as the percent difference between the response to the first block of stimuli and to the last one.

Animals were randomly divided into 5 groups. The following daily treatments initiated immediately before stressor exposure and for a total of four consecutive days: saline, 50 μg/kg ODN1826, 50 μg/kg mEN101, 50 μg/kg μg mEN101 and 500 μg/kg μg ODN2088. Oligonucleotides were delivered in 300 μl 0.9% NaCl solution (Teva, Israel). A control group was exposed to fresh, unused litter. Animals were sacrificed and brains dissected on day 1 2 h after the stressor, on day 4 2 h after the last injection, or on day 7 2 h after the behavioral tests, as noted.

Oligonucleotides

ODN1826 (5′-TCCATGACGTTCCTGACGTT-3′—SEQ ID NO: 1), ODN2088 (5′-TCCTGGCGGGGAAGT-3′—SEQ ID NO: 2), ODN TTAGGG (5′-TTTAGGGTTAGGGTTAGGGTTAGGG-3′—SEQ ID NO: 3) (bases in capital letters are phosphorothioated) were purchased from Invivogen (CA, USA). mEN101 (5′-ctgcaatattttcttgca*c*c*-3′—SEQ ID NO: 4) (stars denote 2′-O-methyl groups), complementary to a sequence in exon E2 of mouse acetylcholinesterase (AChE) mRNA was from Mycrosynth (Balgach, Switzerland). mEN101 was kept frozen at −20° C. in a 10 mM stock concentration until use.

Real-time PCR (RT-PCR): Brains were dissected and tissues stored frozen at −70° C. until use. Hippocampal RNA was extracted using the RNeasy Tissue Mini Kit (Qiagen, Germany). Total RNA was reverse-transcribed (200 ng) using Impromp-II reverse transcriptase (Promega, Madison, Wis.) as instructed. RT-PCR was performed using Power SYBR®Green PCR Master Mix (Applied Biosystems, UK) in a 7900 HT Sequence Detection System (Applied Biosystems, Foster City, Calif.). Quantification was assessed at the logarithmic phase of the PCR reaction. The PCR annealing temperature was 60° C. for all primer pairs. GAPDH was used as a house-keeping gene. The primer pairs employed (Sigma) and the corresponding accession numbers in the GeneBank are presented in Table 1, herein below.

TABLE 1

| | | Product |
|---|---|---|
| Gene | Primers | Size |
| Adenylate cyclase | F(658): TGGAAGCCTGCTC | (187) |
| activating | AAATAGG-SEQ ID NO: 5 | |
| polypeptide 1 | R(845): ATCGAAGTAATGG | |
| receptor 1 | GGGAAGG-SEQ ID NO: 6 | |
| (ADCYAP1R1; | | |
| gi\|70780369) | | |
| calcium/calmodulin- | F(361): TATCGTCCGACTC | (221) |
| dependent protein | CATGACA-SEQ ID NO: 7 | |
| kinase 2 α | R(582): AGGCCAGCAACA | |
| (CAMK2A; | GATTCTC-SEQ ID NO: 8 | |
| NM_177407) | | |
| Nicotinic receptor | F(1008): TGGTGGCAAAAT | (203) |
| subunit α7 | GCCTAAG-SEQ ID NO: 9 | |
| (CHRNA7; | R(1211): CTCGGAAGCCAAT | |
| NM_007390) | GTAGAGC-SEQ ID NO: 10 | |
| Corticotropin | F(475): ATCACCCTCTGCC | (215) |
| releasing hormone | CACTATG-SEQ ID NO: 11 | |
| binding protein | R(690): CTGCAGTTTTGGT | |
| (CRHBP; | GCTGGTA-SEQ ID NO: 12 | |
| gi\|142352317) | | |
| GABA-A receptor | F(706): CATGACAATGCCA | (256) |
| subunit α2 | AACAAGC-SEQ ID NO: 13 | |
| (GABRA2; | R(962): ACTGGCCCAGCAA | |
| NM_008066) | ATCATAC-SEQ ID NO: 14 | |
| Glyceraldehyde-3- | F(1242): GGGTGTGAACC | (176) |
| phosphate | ACGAG-SEQ ID NO: 15 | |
| dehydrogenase | R(1417): AGGGGCCATCCA | |
| (GAPDH; | CAGTCT-SEQ ID NO: 16 | |
| NM_017008) | | |
| Protein kinase C, | F(1744): TGACGAATCACCC | (184) |
| beta | ATTGAAA-SEQ ID NO: 17 | |
| (PRKCB; | R(1928): GTGGCAAGCTGCT | |
| NM_172255.2) | TCTTTCT-SEQ ID NO: 18 | |

Immunohistochemistry: Mice were euthanized on day 1, 2 hours after the initial insult, or 2 hours after the EPM and ASR tests on day 7, by an intraperitoneal injection of 0.1 ml 4% chloral hydrate. Brains were fixed by transcardial perfusion with ice-cold 4% paraformaldehyde containing 4% sucrose (pH=7.4). Immuno-staining was performed using rabbit anti c-fos at 1:5000 dilution (Sigma) or rabbit anti-egrl at 1:1000 dilution (Cell Signaling Technology) followed by biotinylated donkey anti-rabbit at 1:400 dilution (Chemicon) followed by horseradish peroxidase-labeled extravidin at 1:200 dilution (Sigma). Color reaction was produced using diaminobenzidine at a concentration of 0.025% and ammonium nickel sulfate at a concentration of 0.05% and hydrogen peroxide at a concentration of 0.0015%. Two to three coronal brain sections per animal were sampled. Images of each subregion were analyzed using ImagePro Plus software V.4.5.1.29 (Media Cybernetics, Inc., Bethesda, Md.).

Human patient recruitment: PTSD patients and apparently healthy volunteers were recruited from the psychiatric outpatient clinics at various hospitals; PTSD diagnosis was ascertained by a board certified psychiatrist (HS) and scored according to the Clinician-Administered PTSD Scale (CAPS) (Blake, D. D., et al., (1995) J Trauma Stress 8, 75-90). Following recruitment to the study and diagnosis, full medical and psychiatric histories were obtained. Approximately 5 ml of whole samples blood were drawn into heparin-coated plastic tubes. Tubes were placed on ice immediately and serum was separated from cells by centrifugation (4000 rpm, 4° C., 30 minutes). Serum was withdrawn and was stored at −70° C. until use.

Statistics: Statistical comparisons were made by using unpaired Student's t-test, Mann-Withney U-test, ANOVA, and Tukey post-hoc test. Cases showing absolute standard scores>2 were not included in the analyses. Significance was determined at the level of p<0.05.

### Example 1

### Peripheral TLR9 Activation Ameliorates the Long-Term Impact of Traumatic Events

To challenge the hypothesis that TLR9 activation modulates psychological stress reactions, mice were exposed to the predator odor stress paradigm (Cohen, H., Zohar, J., Matar, M. A., Kaplan, Z. & Geva, A. B. (2005) Biol Psychiatry 58, 640-50) and treated them with TLR9 activating or blocking oligonucleotides and with a known stress-suppressing oligonucleotide, mEN101. One week post-exposure, treated mice were tested for avoidance behavior and hyper-arousal in the elevated plus maze (EPM) and acoustic startle response (ASR) tests, respectively (FIG. 1B). Stressed FVB/N mice (n=38) predictably exhibited a higher EPM score than their naïve peers (n=11) (F_{5,103}=21.88, p<0.0001, p<0.0001, one-way ANOVA and Tukey post-hoc test). They also presented greater startle amplitude (F_{5,103}=10.75, p<0.0001; p<0.0001) and decreased startle habituation (F_{5,103}, p<14.19; p<0.0001) than control mice (FIG. 1C). The traumatic experience conferred varied impacts in the exposed mice, ranging from drastically maladaptive behavior to generally unmodified response patterns.

When intraperitoneally injected for 4 consecutive days with 50 μg/kg of the established TLR9 activator ODN1826, mice showed increased stress-induced anxiety scores compared to saline treated controls (n=15; p<0.0001, p<0.419, p<0.04, Tukey post-hoc test for EPM score, startle response and habituation to startle, respectively compared to stressed mice. FIG. 1C). To test if the observed anxiogenic effect was dose-dependent, the dose of ODN1826 was reduced by 100-fold. Such a low dose was reported to slightly affect serum inflammatory markers and leukocyte distribution (Krieg, A. M., Efler, S. M., Wittpoth, M., Al Adhami, M. J. & Davis, H. L. (2004) J Immunother 27, 460-71), unlike the 100-fold higher dose which induces robust inflammatory effects (Krieg, A. M. (2006) Nat Rev Drug Discov 5, 471-84). At 0.5 μg/kg, ODN1826 diminished stress-induced anxiety scores compared to stressed mice (n=11; p<0.04, p<0.02 p<0.078, for EPM score, startle response and habituation to startle response, respectively compared to stressed mice. FIG. 1D), compatible with the different, and sometimes opposite, physiological effects reported for varying concentrations of TLR9 agonists.

Next, mEN101, a molecule shown to ameliorate psychological stress reactions under different paradigms was examined. mEN101 is targeted to the murine acetylcholinesterase (AChE) mRNA and suppresses AChE levels in cultured cells and live animals. The following experiment was performed in order to ascertain whether mEN101 behaves like other TLR9 activators.

Daily intraperitoneal injections of 50 μg/kg mEN101, a dose which would be anxiogenic in ODN1826, diminished stress-induced anxiety scores compared to stressed mice (n=27; p<0.0001, p<0.011, p<0.002, for EPM score, startle response and habituation to startle response, respectively compared to stressed mice. FIG. 1E). To test if this anxiolytic effect was due to TLR9 activation, mEN101 was co-administered with excess (500 μg/kg) of the specific TLR9-inhibitor ODN2088 (n=7). This abolished much of the mEN101 anxiolytic effects, with co-treated mice showing larger EPM scores (p<0.01 Tukey post-hoc test) and smaller habituation to acoustic startle than those of mEN101-treated mice (p<0.015. FIG. 1F). The inhibition of mEN101 anxiolytic effects by TLR9 blockade made the AChE mechanism unlikely as the cause for most of these effects. Intriguingly, the startle response remained unchanged in co-treated mice.

The standard scores of the three behavioral parameters were averaged into a single variable and were denominated anxiety index (AI). AI was higher in stressed mice relative to controls (F_{5,103}=29.69 p<0.0001, p<0.0001, one-way ANOVA and Tukey post-hoc test, FIG. 1G). While the high dose of ODN1826 significantly increased AI compared to saline injected mice (p<0.0001), both ODN1826 at the low dose and mEN101 decreased it (p<0.0001, p<0.00001, respectively, compared to saline injected mice). Furthermore, ODN2088 abolished mEN101 ameliorative effect (p<0.009 compared to mEN101 treated mice). Together, these results suggest that moderate, but not intense TLR9 activation can ameliorate much of the delayed behavioral effects of acute stress.

### Example 2

### mEN101 is a Moderate TLR9 Agonist

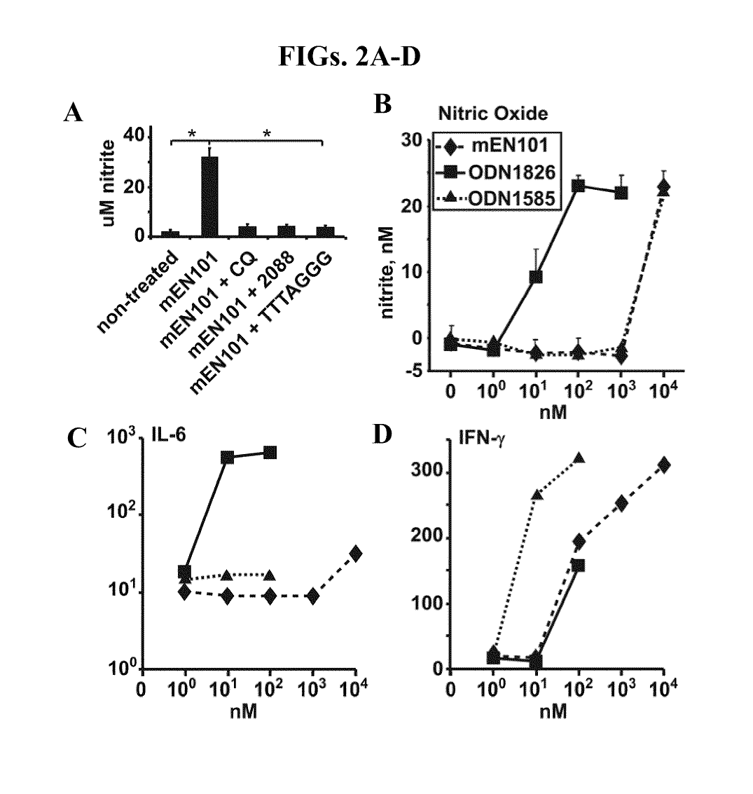

To further characterize the TLR9 activating features of mEN101, mEN101 was applied to RAW264.7 cells (n=4 repeats) for 24 hours and the subsequent production of inflammatory markers was quantified. At 10, but not 0.1 μM, mEN101 increased nitric oxide (NO) production was observed compared to naïve cells (p<0.015, Mann-Whitney test), reflecting weak inflammatory activity. Addition of chloroquine (CQ), a weak base that perturbs endosomal pH and thus disturbs endosomal maturation, as well as the specific TLR9 inhibiting oligonucleotides ODN2088 and ODN TTAGGG (17), all blocked the 10 μM mEN101-driven increases in NO (p<0.015) (FIG. 2A). Thus, both gain and loss of function tests validated the nature of mEN101 as an endosomally active TLR9 activator, albeit a weak one.

Currently reported TLR9 agonists subclassify into three major types. Type-A oligonucleotides induce type-1 interferon (IFN 1) secretion from dendritic cells by interferon regulatory factor (IRF) 7-mediated pathways, activating natural killer cells, macrophages and cytotoxic T cells. Type-B agonists mainly activate NF-κB transcription pathways, leading to the production of cytokines such as IL4, -6, -10 and TNF-α and inducing B cell maturation and antibody secretion. Type-C agonists induce combined effects of type-A and type-B ones (FIG. 1A). In RAW264.7 cells, the type-B ODN1826 induced massive NO production at concentrations as low as 10 nM, (p<0.015), reaching saturation levels at 100 nM. In contrast, parallel doses of the type-A ODN1585 and mEN101 remained totally ineffective in this test; and 10 μM doses were needed to initiate detectable NO overproduction by these oligos (p<0.015) (FIG. 2B). Parallel response curves were observed for TNF-α and IL-6 (FIG. 2C and data not shown), with the maximal mEN101 response being weaker than that of ODN1826 and yet weaker than ODN1585 (n=3 reproducible repeats). At 10 and 100 nM, ODN1826 induced many-fold increases in the mRNA transcripts of the NF-κB activated pro-inflammatory cytokines IL-1α, IL-1β, IL-6, IL-12, and of the anti-inflammatory cytokine IL-10, whereas mEN101 showed much less efficient up-regulation even at high concentrations (see Table 2, herein below).

TABLE 2

| | mEN101 | mEN101 | mEN101 | ODN1826 | ODN1826 | LPS |
|---|---|---|---|---|---|---|
| log2 from NT | 10 nM | 100 nM | 10 uM | 10 nM | 100 nM | 1 ug/ml |
| IL1a | 0.96 | 0.79 | 2.48 | 4.18 | 5.33 | 8.86 |
| IL1b | 1.75 | 1.55 | 3.58 | 4.40 | 5.63 | 5.42 |
| IL6 | 1.65 | 1.29 | 3.68 | 3.57 | 3.71 | 3.81 |
| IL10 | 1.94 | 0.89 | 4.25 | 3.74 | 4.28 | 3.20 |
| IL12 | 0.84 | 1.40 | 2.26 | 3.57 | 4.28 | 8.75 |
| ICAM-1 | −0.46 | −0.84 | −2.03 | −3.57 | −3.14 | −3.74 |

Table 2 illustrates how TLR9 agonists activate inflammatory genes in dose-dependent manner. Shown are fold differences in RT-PCR products of RAW264.7 cells incubated with the noted doses of mEN101 or ODN1826. Note that ODN1826 affects IL1α, -1β, -6, -10, -12 and ICAM-1 gene expression at concentrations as low as 10 nM, whereas considerably higher doses of mEN101 are needed to attain similar effects. Nevertheless, mEN101 affects inflammatory gene expression even at 10 nM. 1 μg/ml LPS was used as positive control. (n=3, p<0.05 Mann-Whitney U-test compared to untreated controls, for all the data shown).

Induction of the Th2 cytokine IFN-γ in splenocytes was predictably stronger for ODN1585 than ODN1826, and mEN101 induced considerably weaker IFN-γ responses even at 10 μM doses (FIG. 2D). Thus, mEN101 showed TLR9 activating effects at wide range concentrations, but it was consistently less effective than ODN1826.

### Example 3

### TLR9 Signaling Abolishes Delayed Stress-Induced Hyper-Activation of Brain Nuclei

Acute psychological stressors increase the activity of several stress-related nuclei in the brain. Compatible with this, cat odor-stressed FVB/N mice (n=3) showed increased c-fos staining 2 hours following stress exposure compared to naïve controls (FIGS. 3B-C) in the hypothalamic paraventricular nucleus (PVN), the hippocampal CA3 region, and the amygdala (AMG) (p<0.05 for all, Mann-Whitney U-test) amongst other brain areas. A single mEN101 dose administered immediately before the stressor did not modify this response (FIG. 3D), suggesting that TLR9 activation by mEN101 does not diminish the threat perception and the acute reaction induced by potent stressors. Similarly, the immediate c-fos upregulation induced by immobilization stress was not modified by mEN101 treatment (data not shown). To test if this early reaction to the acute stressor is followed by a subsequently accentuated stress response, animals were exposed to predator odor with or without mEN101 treatment. One week later the EPM and ASR tests were performed. The animals were sacrificed 2 hours later. Pre-stressed mice (n=3) showed enhanced neural activity, reflected by c-fos immunohistochemistry, compared to naïve controls (n=4). c-fos enhancement spanned the PVN, CA3 and AMG (p<0.02 for all), amongst other brain areas. Importantly, TLR9 activation by mEN101 (n=4) restricted these delayed c-fos increases (p<0.05 for all) (FIG. 3F). Thus, moderate TLR9 activation maintained the immediate alertness during acute stress, yet avoided its later over-reactive consequences.

### Example 4

### Stress-Controlling TLR9 Stimulation is Anti-Inflammatory

Stressful sensory stimuli are processed, and consequent behavioral stress responses are initiated in the brain. That peripheral TLR9 activation effectively suppressed both the delayed neuronal c-fos induction and the associated behavioral post-traumatic effects indicated the existence of an inherent negative-feedback circuit from blood to brain and back. The brain identifies aversive sensory stimuli and sends stress messages to the periphery, through the sympathetic system and the HPA axis. Subsequent increases of diverse pro-inflammatory cytokines were reported at the central and peripheral levels (Nguyen, K. T., Deak, T., Owens, S. M., Kohno, T., Fleshner, M., Watkins, L. R. & Maier, S. F. (1998) J Neurosci 18, 2239-46). Correspondingly, stressed mice (n=4) showed prolonged increases in the serum levels of IL-6 (p<0.02, Mann-Whitney test) with a parallel trend in IL-1β (p<0.15) 4 days following exposure, compared to naïve animals (n=5, FIGS. 4A-B). Peripherally-produced cytokines are brain-penetrant and induce over-stimulation of interleukin receptors located in diverse hypothalamic and extra-hypothalamic areas. Importantly, intraperitoneal treatment with mEN101 completely abolished these prolonged stress-induced increases in IL-1β (n=4, p<0.02) and IL-6 (p<0.004) (FIGS. 4A-B). This suggested interference with the enhancement of anxiety behavior by direct stimulation of hypothalamic and extra-hypothalamic stress-related nuclei, and correspondingly diminished consolidation of fear memories.

### Example 5

### TLR9 Controls Delayed Gene Expression Changes in Stress-Responding Brain Regions

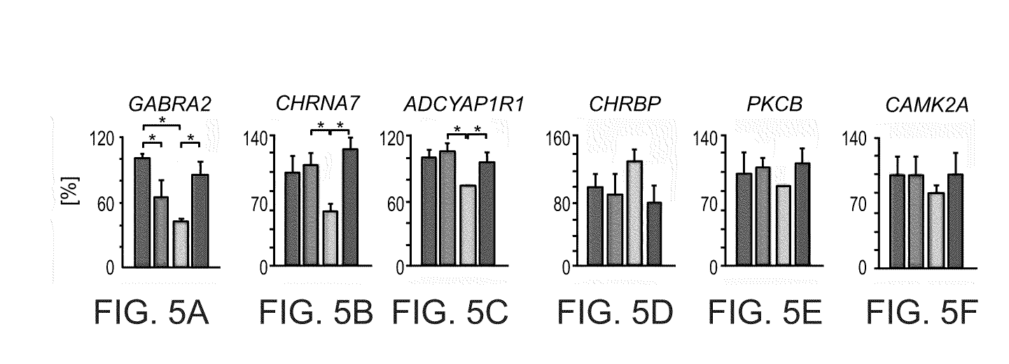

The transcription factor early growth response gene 1 (egr-1) is a central mediator of inflammation and is broadly and constitutively expressed in the central nervous system (CNS). Egr-1 expression increases during stressful situations, affecting long-term fear-learning processes and long-term potentiation (LTP) by regulating down-stream late-response genes. Pre-stressed mice showed increased egr-1 expression compared to naïve mice in brain nuclei expressing the IL-1β and IL-6 receptors (e.g., CA3 and AMG), as well as in the PVN. Peripheral TLR9 activation by mEN101 abolished these increases in PVN and AMG, but not in the CA3 region (FIG. 4E). This suggested that peripheral modulation of pro-inflammatory cytokines by TLR9 may selectively limit their excessive signaling to the CNS, preventing the activation of interleukin receptors and subsequent stress-induced consequences. Therefore, the present inventors searched for modifications in anxiety-related transcripts in the hippocampus, a major structure of the limbic system. The Mouse Genome Informatics (MGI, worldwidewebdotinformaticsdotjaxdotorg/) initiative reports 259 genes related to anxiety phenotypes. Of those, 11 genes were selected that were expressed in the hippocampus and reported to be involved in abnormal EPM reactions. Hippocampi of naïve and stressed mice, mEN101-treated or untreated, were tested 4 days following stress exposure (n=4 each group). Stress alone reduced hippocampal GABRA2 mRNA levels, encoding for the GABA receptor α2 subunit (GABRA2) (p<0.001, one-tailed t-test), which mediates benzodiazepine anxiolytic effects. Following TLR9 activation by mEN101 treatment, stressed hippocampi showed further reduction in GABRA2, accompanied by reduced nicotinic receptor subunit α7 (CHRNA7) and adenylate cyclase activating polypeptide 1 receptor 1 (ADCYAP1R1) levels compared to untreated stressed mice (p<0.03 for all) (FIGS. 5A-F). This suggested decreased cholinergic and pituitary gland activation, both mediators of psychological stress responses. Inhibiting TLR9 activation by co-administration of ODN2088 abolished the mEN101-induced modulation of these genes, with a parallel trend in the anxiety-related genes corticotrophin releasing hormone binding protein (CHRBP), which controls HPA axis activation, and in the fear-conditioning related genes protein kinase Cβ (PKCB), and calcium/calmodulin-dependent protein kinase 2α (CAMK2A) (p<0.03 for all) (FIGS. 5A-F).

### Example 6

### TLR9 Ablation Changes Anxiety-Related Behavior

That manipulating TLR9 activity could mitigate the impact of traumatic memories suggested that inherited differences in TLR9 actions might affect general anxiety features. To challenge this prediction, C57/B6 TLR9 −/− mice with genetically ablated TLR9 (Hemmi, H., et al., (2000) Nature 408, 740-5) were examined and their behavior was compared to that of strain-matched controls under baseline and stress conditions. One week post-stress, control C57/B6 mice (n=16), similar to FVB/N mice, showed enhanced anxiety reactions compared to naïve controls (n=10): they exhibited higher EPM score (F_{4,35}=34.3, p<0.0001, one-way ANOVA; p<0.0001, Tukey post-hoc test), greater mean startle amplitude (F_{4,35}=8.05, p<0.0001; p<0.0001) and lower startle habituation (F_{4,35}=21 p<0.0001; p<0.0001) compared to naïve peers. Importantly, TLR9 −/− naïve mice showed increased baseline anxiety features compared to controls, accompanied by failure to fully react to cat odor stress (FIG. 6A-D). Specifically, naïve TLR9 −/− mice (n=6) exhibited increased EPM score (p<0.01, Tukey post-hoc test), reflecting greater basal anxiety than controls. However, their startle response and startle habituation were similar to C57/B6 mice, suggesting intact motor instincts and habituation learning capacities. Unlike the accentuation of their basal anxiety phenotype, TLR9 −/− mice were conspicuously less reactive to predator odor stress than C57/B6 controls. One week following such stress, TLR9 −/− mice presented increased EPM score (n=3, p<0.03, Mann-Whitney U-test). Additionally, TLR9 −/− mice experienced somewhat reduced startle amplitude, suggesting hypo-sensitized state, relative to stressed C57/B6 mice. In contrast, startle habituation was fully conserved in TLR9 −/− mice (FIG. 6D). After-stress variations in the measured parameters were insignificant in TLR9 −/− relative to C57/B6 controls (p<0.09, p<0.156, p<0.39, Mann-Whitney U-test for EPM score, startle response and startle habituation).

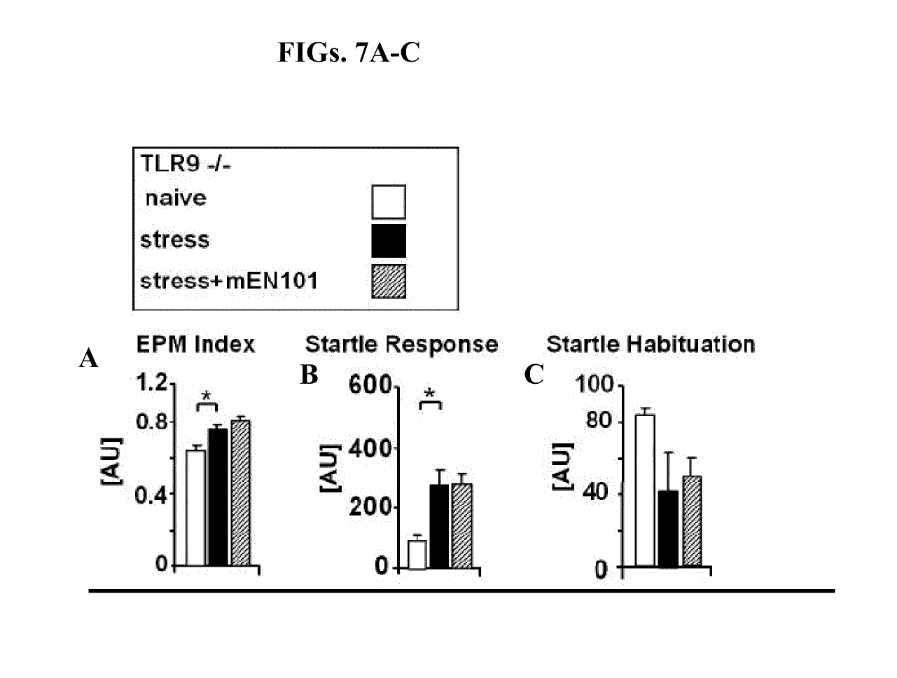

Next, TLR9 −/− mice (n=3) were intraperitoneally injected daily with 500 μg/kg mEN101, starting immediately after the stressor and for a total of four consecutive days. Such dose was recently found to be anxiolytic in a similar PTSD model (Adamec, R., et al., (2008) Behav Brain Res 189, 180-90). Unlike FVB/N and C57/B6 mice, TLR9 −/− mice were totally refractory to mEN101, which changed none of the observed parameters (FIGS. 7A-C).

AI was higher in stressed mice relative to controls (F_{3,99}=8.99 p<0.0001, p<0.0001, one-way ANOVA and Tukey post-hoc test, FIG. 6E). TLR9 −/− naïve mice exhibited increased AI relative to WT controls, though the difference was insignificant (p<0.47). Interestingly, the AI of stressed TLR9 −/− was not significantly increased relative to non-exposed TLR9 −/− controls and, as expected, mEN101 did not affect TLR9 −/− response to the acute stressor (FIGS. 7A-C).

Together, the present findings support the notion that endogenous TLR9 activation conveys constant peripheral signals which constrain basal stress-responsive behavior and modulate the processing of psychologically aversive stimuli in the brain. To further explore this possibility, anxiety-related transcripts in hippocampi from naïve TLR9 −/− were tested and compared to naïve C57/B6 mice (n=6, in each group). Up-regulation of GABRA2 and down-regulation of CHRBP (p<0.01, one-tailed t-test) both predicted increased HPA axis activation. Parallel decreases in PKCβ transcripts (p<0.05) suggested impaired contextual fear learning, such as cued and fear conditioning (25), compatible with the decreased anxiety phenotype of stressed TLR9 −/− mice in hippocampus-related tasks. Other tested stress-related transcripts (e.g. CHRNA7 and CAMK2A) were unaltered in TLR9 −/− hippocampi, attesting to the selectivity of the observed differences (FIGS. 6F-J).

### Example 7

### Symptoms Severity and Hippocampal Volume Decrease in PTSD Patients Correlate with Serum Inflammatory Markers

To find out if stress-induced inflammation is also relevant to long-lasting stress responses in human PTSD patients, IL1β and IL6 levels were measured in the serum of male patients and healthy, age-matched controls. IL1β was increased in PTSD patients (n=22, p<0.016, two-tailed t-test). IL6 showed a similar although insignificant trend, perhaps due to the small cohort (FIGS. 8A-B). Nevertheless, the summated IL1β and IL6 concentration in serum (designated “inflammatory load”) was positively correlated with PTSD symptoms severity, as assessed by the Clinician-Administered PTSD Scale (CAPS), which is composed of re-experience, avoidance and arousal scores, each assessing a major symptomatic axis of PTSD, the Montgomery-Åsberg Depression Rating Scale (MADRS) and the Pittsburgh Sleep Quality Index (PSQI). Next, PTSD patients were divided into high and low inflammatory load groups (FIGS. 8A-C). Patients with high inflammatory load (n=9) showed increased re-experiencing (p<0.014, FIG. 8D), arousal (p<0.022, FIG. 8E) and MADRS (p<0.02, FIG. 8F) scores compared to PTSD patients with low inflammatory load (n=10). A similar trend, yet insignificant, was observed for the overall CAPS score (FIG. 8G), the avoidance score (FIG. 8I), and the PSQI (FIG. 8H) (p<0.058, p<0.19 and p<0.24 respectively). IL1β values which were significantly higher in PTSD patients were then correlated with neuroanatomical features characteristic of PTSD. Reduced hippocampal volume, an established neuroanatomical marker of PTSD, was inversely correlated to IL1β in PTSD patients serum (n=5, r=−0.920, p<0.006 and r=−0.794, p<0.022, Pearson correlation for right and left hippocampus respectively, FIGS. 8J-K). No correlation was found between IL1β and the volume of other brain structures, such as cerebral cortex, amygdale, thalamus, pallidum, brain stem and ventricles (FIGS. 8J-K and data not shown). The correlation between peripheral inflammatory cytokines and psychiatric and physiological PTSD biomarkers, together with the results obtained in mice, postulate stress-induced inflammation as an etiological component of PTSD pathology.

### Discussion for Examples 1-7

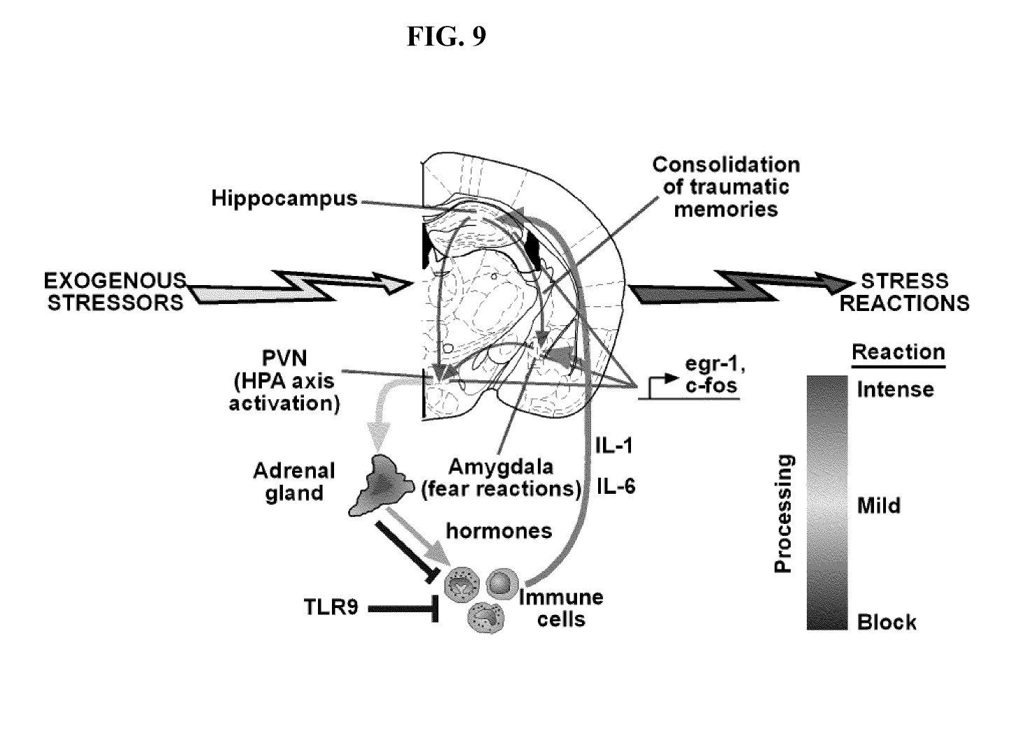

The present inventors found that inherent TLR9 signaling modulates both basal and stressor-induced anxiety, whereas transient and moderate TLR9 activation exerts long-lasting suppression of pro-inflammatory cytokines accompanied by blockade of stress-inducible activities in responding brain nuclei (FIG. 9). The suppressed anxiety and diminished neural reactions to stress of mice moderately treated with TLR9 agonists, suggest that manipulating TLR9 signaling can modulate the capacity to acquire and/or consolidate stress memories. Furthermore, the prominent correlations that were found in PTSD patients between hippocampal volume decrease, PTSD severity scores and inflammatory load likewise strengthen the hypothesis that monitoring inflammatory reactions can mitigate delayed stress responses in humans.

Immune system receptor(s) capable of conveying threat signals to the brain are evolutionarily advantageous, but continuous propagation of cytokine/stress signals may be damaging. Therefore, refined monitoring of such propagation, e.g. by TLR9, can support balanced functioning, provided that its stimulatory activity is moderate. Compatible with this prediction, peripheral and moderate stimulation of TLR9 by both the type-B ODN1826 and the non-CpG oligo mEN101 ameliorated the delayed behavioral effects of acute stress. This suggests that the psycho-active effects of TLR9 ligands initiate in TLR9-expressing leukocytes and that the observed IL-1β and IL-6 suppression is one of the first steps in the anxiolytic cascade.

Pro-inflammatory cytokines are good candidates for modulating the TLR9 stress-suppressive effects, since even slight increases in them enhance anxiety behavior, both by directly stimulating hypothalamic and extra-hypothalamic stress-related nuclei and by facilitating the consolidation of fear memories. Suppression of cytokine levels is hence likely to limit the impact of stress, compatible with the anxiolytic dose-dependent effect of TLR9 activation: both high doses of mEN101 and low doses of ODN1826 ameliorated the behavioral outcomes of acute stress. Inversely, high doses of ODN1826, known to induce peripheral IL-1β and IL-6 (Krieg, A. M. (2006) Nat Rev Drug Discov 5, 471-84) and to activate stress-related nuclei (Sako, K., Okuma, Y., Hosoi, T. & Nomura, Y. (2005) J Neuroimmunol 158, 40-9), yielded a slight anxiogenic effect, compatible with the phenomenon of “sickness behavior”. That ODN1826 exerts stress-suppressive activity at 100-folder lower dose than that enhancing immune function may imply that activating only part of the TLR9 molecules in target cells or tissues is necessary and sufficient to induce this monitoring activity.

TLR9 −/− mice showed a bimodal behavioral imbalance compared to their strain-matched controls: increased basal anxiety and diminished reaction to acute stressors. This unusual functioning phenotype could be attributed either to impaired CNS development or to a sustained failure of stress-induced pathways. The selective differences in hippocampal gene expression of TLR9 −/− mice, in conjunction with these behavioral characteristics, support the latter option: that TLR9 functioning conveys constant peripheral signals which constrain basal stress-responsive behavior while modulating the processing of psychologically aversive stimuli in the CNS. That TLR9 −/− naïve mice showed increased basal EPM score, likely reflects modified hippocampal-dependent contextual decision making and spatial recognition of safer and threatening areas. Such behavior is often associated with modified functioning of the ventral angular bundle (VAB)—basolateral amygdala (BLA) pathway. In contrast, the basal performance of TLR9 −/− mice in the ASR paradigm, which involves increased central amygdala (CEA)—periaqueductal gray matter (PAG) connectivity was indistinguishable from that of strain-matched controls. Likewise, startle habituation was fully conserved in TLR9 −/− mice. This latter reaction stems from the cochlear nucleus to reach the caudal pontine reticular nucleus (PNC) and elicits the motor responses via direct output to motor neurons, leading to synaptic depression which is apparently unrelated to the TLR9 response. The distinct performance of TLR9 −/− relative to WT mice in the EPM, ASR and startle habituation paradigms thus suggests that specific internal brain circuits are affected by physiological TLR9 activation.

At another level, the present findings attribute the decreased peripheral AChE levels under mEN101 to TLR9-mediated pathway(s), suggesting that moderate TLR9 activation leads to enhanced peripheral cholinergic signaling. That such signaling further suppresses inflammatory reactions suggests the existence of a positive feedback loop, whereby TLR9 augments cholinergic signals which in turn mitigate inflammatory cytokines and accentuate the suppression of stress reactions.

Together, the results show that TLR9 monitors brain-signaling cytokines in a manner which is both necessary and sufficient for minimizing the behavioral and cognitive consequences of acute stress responses. Moreover, it has been demonstrated here that the accuracy of such monitoring is amenable for inherited changes as well as therapeutic manipulations.

### Example 8

### TLR9 Agonists Increase Glucocorticoid Anti-Inflammatory Effect by Up-Regulating HSP90

The present example communicates that pre-treating toll-like receptor 9 (TLR9)-expressing cells with TLR9 agonists increase glucocorticoid (GC) binding to glucocorticoid receptor (GR) and potentiates GC anti-inflammatory effect in leukocytes.

Materials and Methods

Oligonucleotides: ODN 1826 (5′-TCCATGACGTTCCTGACGTT-3′ phosphorothioated backbone—SEQ ID NO: 1) was purchased from Invivogen (CA, USA). mEN101 is a 20-mer oligonucleotide (5′-CTGCAATATTTTCTTGCA*C*C*-3′, SEQ ID NO: 4, stars denote 2-O-oxymethyl groups) and was synthesized by Mycrosynth (Switzerland) and kept in 10 mM stock concentration. hEN101 is a 20-mer oligonucleotide (5′-CTGCCACGTTCTCCTGCA*C*C*-3′ SEQ ID NO: 20, stars denote 2-O-oxymethyl groups) and was synthesized by Hybridon, Inc. Worchester, U.S.A.).

Cell cultures: The mouse macrophage cell line RAW 264.7 (ATCC, MD) was cultured in Dulbecco's modified Eagle's medium (DMEM) (Biological Industries, Israel) supplemented with 10% heat-inactivated fetal bovine serum (Biological Industries, Israel), 100 μg/ml streptomycin (Sigma), 100 U/ml penicillin (Sigma) and 2 mM 1-glutamine (Biological Industries, Israel) at 37° C. in a humidified atmosphere with 5% CO₂.

Animals: The stress paradigm consisted of placing the test animals on well-soiled cat litter for 10 minutes (in use by the cat for 2 days, sifted for stools). Tissues were removed 4 days following exposure.

Real-time PCR (RT-PCR): RAW264.7 cells and spleen were used for RT-PCR. 1×10⁶ RAW264.7 cells were grown in 2 ml medium in 6 wells plates until 80% confluence was obtained. Total RNA was extracted using the RNeasy kit (Qiagen, CA, USA). Spleen and brain tissues' RNA was extracted using the RNeasy Tissue Mini Kit (Qiagen, Germany). In all cases, total RNA was reverse-transcribed (200 ng) using Impromp-II reverse transcriptase (Promega, WI, USA) according to manufacturer's instructions. RT-PCR was performed using Power SYBR®Green PCR Master Mix (Applied Biosystems, UK) in a 7900 HT Sequence Detection System (Applied Biosystems, CA). Quantification was assessed at the logarithmic phase of the PCR reaction. The PCR annealing temperature was 60° C. for all primer pairs.

Immunohistochemistry: Cells were fixed with 4% PFA in phosphate-buffered saline (PBS) for 15 minutes at room temperature (RT), washed and transferred to 100% MetOH at −20° C. for 5 minutes. Afterwards, cells were washed twice with a blocking medium containing PBS, 1% bovine serum albumin (BSA) and 0.01% NaN₃ and then were incubated for 1 hour at RT with anti-HSP90 and anti-GR antibodies (1:50) (Santa Cruz Biotechnology, CA). After incubation, cells were washed twice in blocking medium and incubated with 1:100 secondary antibodies (Jackson Immunoresearch Laboratories, PA). Slides were mounted onto glass slides and visualized by confocal microscopy.

Glucocorticoid receptor assay: A whole cell binding assay was used to determine GR number and affinity. 0.7×10⁶ RAW264.7 cells in 2 ml medium were grown in 24-wells plates until 80% confluence was obtained. After the appropriate treatment, the medium was replaced with a saturating concentration of 50 nM [³H]dexamethasone (85.0 Ci/mmol) (Amersham Biosciences, NJ, USA) in 5 μM unlabeled dexamethasone (Sigma, Israel). After incubation for 2 hours at 37° C., cells were washed 6 times with cold PBS and lysed in 1M NaOH. Lysates were harvested and counted in a beta-spectrometer.

Determination of nitric oxygen (NO) production: The nitrite concentration in the medium was measured as an indicator of NO production according to the Griess reaction. 1×10⁵ RAW264.7 cells were grown in 0.5 ml medium in 48-wells plate until 80% confluence was obtained. 100 ml supernatant was incubated with 100 ml Griess reagent (1% sulfanilamide, 0.1% naphthylethylenediamine dihydrochloride, 5% H₃PO₄). Absorbance was read at 540 nm after 10 minutes using SPECTRAFluor Plus spectrophotometer (TECAN, UK).

Results

TLR9 activation induces the up-regulation of diverse heat-shock proteins (HSPs) in vitro (e.g., HSP90). Correspondingly, 24 hour incubation with either ODN1826 (SEQ ID NO: 2) or mEN101 (SEQ ID NO: 4) significantly increased HSP90β mRNA levels in RAW264.7 cells (p<0.05 for both, Mann-Whitney test) (FIG. 10B). Stressed mice treated with mEN101 showed increased HSP9013 mRNA levels in spleen (p<0.04, Mann-Whitney test), but not in the frontal lobe (p<0.2, Mann-Whitney test). HSP90β mRNA up-regulation was completely abolished by ODN2088 co-administration (p<0.06, Mann-Whitney test) (FIG. 10B).

Since HSP90 is a molecular chaperone that binds glucocorticoid receptor (GR) in its inactive state, it was reasoned that increased concentrations of HSP90 may increase the number of GR complexed with it. Indeed, hEN101-upregulated HSP90, but not GR, in a dose dependent manner in RAW264.7 cells. Moreover, such increases were accompanied by increased colocalization of HSP90 and GR signals. (FIG. 10C)

Since HSP90 modulates GR activity by increasing its affinity to glucocorticoids (GCs) it was wondered whether such an effect could be obtained by activating TLR9. In order to challenge that hypothesis, RAW264.7 cells were incubated with varying concentrations of hEN101 for 24 hours and the medium was subsequently replaced with one containing different concentration of 50 nM [³H]dexamethasone and 5 μM unlabeled dexamethasone. After a 2 hour incubation, radioactivity was measured in a beta-spectrometer. TLR9 activation by hEN101 (SEQ ID NO: 20) increased [³H]dexamethasone incorporation to RAW264.7 cells in a dose-dependent manner (R²=0.96), with detectable effects observed from 1 μM concentrations and up (p<0.02, Mann-Whitney test). Scatchard plot analysis revealed that DEX Bmax, but not Kd, was increased, indicating an increased number of GR binding sites (FIG. 10D). Importantly, hEN101 effect was not observed in the myotube cell line C2C12, which do not express TLR9.

In order to study the functional effects of TLR9 activation on GR signaling, RAW264.7 cells were incubated with varying concentrations of ODN1826 (SEQ ID NO: 2) for 24 hours and the the medium was replaced with one containing LPS (1 μg/ml) with or without 10 or 50 mM corticosterone (CORT) for an additional 24 hours. As expected, CORT blocked lipopolysaccharide (LPS)-induced NO production. At low doses (0.01-1 nM), ODN1826 potentiated CORT anti-inflammatory effect. However, at higher doses (10-100 nM) ODN1826 increased cell activation, probably due to its inflammatory effect (FIG. 10E).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of treating an anxiety-related disorder in a subject in need thereof, the method comprising administering to the subject a therapeutically effective amount of a toll-like receptor 9 (TLR-9) agonist, with the proviso that said TLR-9 agonist is not EN101, thereby treating the anxiety-related disorder.

2. A method of treating an inflammatory disorder in a subject in need thereof, the method comprising co-administering to the subject a therapeutically effective amount of a glucocorticoid and a TLR-9 agonist, thereby treating the inflammatory disorder.

3. An article of manufacture comprising a TLR-9 agonist and a glucocorticoid.

4. The method claim 1, wherein said TLR-9 agonist is an oligonucleotide comprising one or more unmethylated CpG dinucleotide (CpG ODNs).

5. The method claim 1, wherein said TLR-9 agonist induces type-1 interferon (IFN1) secretion from dendritic cells.

6. The method claim 1, wherein said TLR-9 agonist upregulates an activity of nuclear factor kappa-light-chain-enhancer of activated B cells (NF-κB).

7. The method of claim 4, wherein said CpG ODN is selected from the group consisting of a type-A CpG ODN, a type-B CpG ODN and a type-C CpG.

8-9. (canceled)

10. The method of claim 1, wherein said administering is effected by systemic administration.

11. The method of claim 1, wherein said administering is not effected directly administering to the brain.

12. The method of claim 10, wherein said systemic administration is selected from the group consisting of oral administration, intravenous (IV) administration, intrarterial (IA) administration, intramuscular (IM) administration, subcutaneous (SC) administration and intraperitoneal (IP) administration.

13. The method of claim 10, wherein said systemic administration is oral administration.

14. The method of claim 4, wherein said CpG ODN comprises ODN1585 (SEQ ID NO: 19) or ODN1826 (SEQ ID NO: 1).

15. (canceled)

16. The method of claim 1, wherein an activity of said TLR-9 agonist is down-regulated by ODN2088 (SEQ ID NO: 2).

17. The method of claim 1, wherein the anxiety-related disorder is post traumatic stress syndrome (PTSD).

18. (canceled)

19. The method of claim 14, wherein a therapeutically effective amount of ODN1826 is from about 0.01 μg/kg to 0.09 μg/kg.

20. The method of claim 19, wherein a therapeutically effective amount of ODN1826 is about 0.04 μg/kg.

21. The method of claim 2, wherein said TLR-9 agonist comprises EN101.

22-23. (canceled)

24. The method of claim 2, wherein said TLR-9 agonist is an oligonucleotide comprising one or more unmethylated CpG dinucleotide (CpG ODNs).

25. The article of manufacture of claim 3, wherein said TLR-9 agonist is an oligonucleotide comprising one or more unmethylated CpG dinucleotide (CpG ODNs).

26. The method of claim 24, wherein said CpG ODN is selected from the group consisting of a type-A CpG ODN, a type-B CpG ODN and a type-C CpG.

27. The article of manufacture of claim 25, wherein said CpG ODN is selected from the group consisting of a type-A CpG ODN, a type-B CpG ODN and a type-C CpG.

28. The method of claim 24, wherein said CpG ODN comprises ODN1585 (SEQ ID NO: 19) or ODN1826 (SEQ ID NO: 1).


SEQUENCE LISTING (28 sequences)

SEQ ID NO: 1 - DNA, 20 bp, Artificial sequence
  CpG ODN 1826
tccatgacgttcctgacgtt

SEQ ID NO: 2 - DNA, 15 bp, Artificial sequence
  ODN2088
tcctggcggggaagt

SEQ ID NO: 3 - DNA, 25 bp, Artificial sequence
  ODN TTAGGG
tttagggttagggttagggttaggg

SEQ ID NO: 4 - DNA, 20 bp, Artificial sequence
  mEN101
ctgcaatattttcttgcacc

SEQ ID NO: 5 - DNA, 20 bp, Artificial sequence
  Single strand DNA oligonucleotide
tggaagcctgctcaaatagg

SEQ ID NO: 6 - DNA, 20 bp, Artificial sequence
  Single strand DNA oligonucleotide
atcgaagtaatgggggaagg

SEQ ID NO: 7 - DNA, 20 bp, Artificial sequence
  Single strand DNA oligonucleotide
tatcgtccgactccatgaca

SEQ ID NO: 8 - DNA, 19 bp, Artificial sequence
  Single strand DNA oligonucleotide
aggccagcaacagattctc

SEQ ID NO: 9 - DNA, 19 bp, Artificial sequence
  Single strand DNA oligonucleotide
tggtggcaaaatgcctaag

SEQ ID NO: 10 - DNA, 20 bp, Artificial sequence
  Single strand DNA oligonucleotide
ctcggaagccaatgtagagc

SEQ ID NO: 11 - DNA, 20 bp, Artificial sequence
  Single strand DNA oligonucleotide
atcaccctctgcccactatg

SEQ ID NO: 12 - DNA, 20 bp, Artificial sequence
  Single strand DNA oligonucleotide
ctgcagttttggtgctggta

SEQ ID NO: 13 - DNA, 20 bp, Artificial sequence
  Single strand DNA oligonucleotide
catgacaatgccaaacaagc

SEQ ID NO: 14 - DNA, 20 bp, Artificial sequence
  Single strand DNA oligonucleotide
actggcccagcaaatcatac

SEQ ID NO: 15 - DNA, 16 bp, Artificial sequence
  Single strand DNA oligonucleotide
gggtgtgaaccacgag

SEQ ID NO: 16 - DNA, 18 bp, Artificial sequence
  Single strand DNA oligonucleotide
aggggccatccacagtct

SEQ ID NO: 17 - DNA, 20 bp, Artificial sequence
  Single strand DNA oligonucleotide
tgacgaatcacccattgaaa

SEQ ID NO: 18 - DNA, 20 bp, Artificial sequence
  Single strand DNA oligonucleotide
gtggcaagctgcttctttct

SEQ ID NO: 19 - DNA, 20 bp, Artificial sequence
  Exemplary CpG ODN (ODN 1585)
ggggtcaacgttgagggggg

SEQ ID NO: 20 - DNA, 20 bp, Artificial sequence
  hEN101
ctgccacgttctcctgcacc

SEQ ID NO: 21 - DNA, 20 bp, Artificial sequence
  rEN101
ctgcgatattttcttgtacc

SEQ ID NO: 22 - DNA, 24 bp, Artificial sequence
  Exemplary CpG ODN (CpG ODN 2006)
tcgtcgttttgtcgttttgtcgtt

SEQ ID NO: 23 - DNA, 20 bp, Artificial sequence
  Exemplary CpG ODN (CpG ODN 2216)
gggggacgatcgtcgggggg

SEQ ID NO: 24 - DNA, 25 bp, Artificial sequence
  Exemplary CpG ODN (CpG ODN M362)
tcgtcgtcgttcgaacgacgttgat

SEQ ID NO: 25 - DNA, 24 bp, Artificial sequence
  Exemplary CpG ODN (CpG ODN 7909)
tcgtcgttttgtcgttttgtcgtt

SEQ ID NO: 26 - DNA, 23 bp, Artificial sequence
  Exemplary CpG ODN
tctgtcgttctntcttgctgtct

SEQ ID NO: 27 - DNA, 23 bp, Artificial sequence
  Exemplary CpG ODN
tctgtcgttctntcttgctgtct

SEQ ID NO: 28 - DNA, 23 bp, Artificial sequence
  Exemplary CpG ODN
tctgtcgttctntcttgctgtct